SEQUENCE LISTING (127 sequences)

SEQ ID NO: 1 - protein, 240 aa, Artificial Sequence
  source: /note=“Description of Artificial Sequence Synthetic polypeptide”
DIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKRGGGGSGGGGSGGGGSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGQGTLVTVSS

SEQ ID NO: 2 - protein, 240 aa, Artificial Sequence
  source: /note=“Description of Artificial Sequence Synthetic polypeptide”
QVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGRGTLVTVSSGGGGSGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKR

SEQ ID NO: 3 - protein, 240 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKGGGGSGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSS

SEQ ID NO: 4 - protein, 240 aa, Artificial Sequence
  source: /note=“Description of Artificial Sequence Synthetic polypeptide”
EVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIK

SEQ ID NO: 5 - protein, 138 aa, Artificial Sequence
  source: /note=“Description of Artificial Sequence Synthetic polypeptide”
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 6 - protein, 146 aa, Artificial Sequence
  source: /note=“Description of Artificial Sequence Synthetic polypeptide”
CYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 7 - protein, 166 aa, Artificial Sequence
  source: /note=“Description of Artificial Sequence Synthetic polypeptide”
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 8 - protein, 220 aa, Artificial Sequence
  source: /note=“Description of Artificial Sequence Synthetic polypeptide”
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDG

SEQ ID NO: 9 - protein, 229 aa, Artificial Sequence
  source: /note=“Description of Artificial Sequence Synthetic polypeptide”
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGS

SEQ ID NO: 10 - protein, 107 aa, Artificial Sequence [sequence omitted]

SEQ ID NO: 11 - protein, 113 aa, Artificial Sequence [sequence omitted]

SEQ ID NO: 12 - protein, 111 aa, Artificial Sequence [sequence omitted]

SEQ ID NO: 13 - protein, 120 aa, Artificial Sequence [sequence omitted]

SEQ ID NO: 14 - protein, 493 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKRGGGGSGGGGSGGGGSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGQGTLVTVSS

SEQ ID NO: 15 - protein, 493 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGRGTLVTVSSGGGGSGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKR

SEQ ID NO: 16 - protein, 493 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKGGGGSGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSS

SEQ ID NO: 17 - protein, 493 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIK

SEQ ID NO: 18 - protein, 380 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 19 - protein, 388 aa, Artificial Sequence
  source: /note=“Description of Artificial Sequence Synthetic polypeptide”
CYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQGGGGSGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGA

SEQ ID NO: 20 - protein, 493 aa, Artificial Sequence
  source: /note=“Description of Artificial Sequence Synthetic polypeptide”
DIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKRGGGGSGGGGSGGGGSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGQGTLVTVSSGGGGSGGGGSGPLGVRGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 493 aa, Artificial Sequence
  source: /note=“Description of Artificial Sequence Synthetic polypeptide”
QVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGRGTLVTVSSGGGGSGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKRGGGGSGGGGSGPLGVRGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 493 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKGGGGSGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSGGGGSGGGGSGPLGVRGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 493 aa, Artificial Sequence
  source: /note=“Description of Artificial Sequence Synthetic polypeptide”
EVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKGGGGSGGGGSGPLGVRGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 379 aa, Artificial Sequence
  source: /note=“Description of Artificial Sequence Synthetic polypeptide”
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGGGGSGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 388 aa, Artificial Sequence
  source: /note=“Description of Artificial Sequence Synthetic polypeptide”
CYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQGGGGSGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 13 aa, Artificial Sequence
  source: /note=“Description of Artificial Sequence Synthetic peptide”
GGSLSGRSDNHGS

SEQ ID NO: 27 - protein, 42 aa, Artificial Sequence
  source: /note=“Description of Artificial Sequence Synthetic polypeptide”
GGGGSGGGGSGGGGSISSGLLSSGGSGGSLSGRSDNHGGGGS

SEQ ID NO: 28 - protein, 6 aa, Artificial Sequence
  source: /note=“Description of Artificial Sequence Synthetic peptide”
GPLGVR

SEQ ID NO: 29 - protein, 7 aa, Artificial Sequence
  source: /note=“Description of Artificial Sequence Synthetic peptide”
PLGMWSR

SEQ ID NO: 30 - protein, 7 aa, Artificial Sequence
  source: /note=“Description of Artificial Sequence Synthetic peptide”
PLGLWAR

SEQ ID NO: 31 - protein, 8 aa, Artificial Sequence
  source: /note=“Description of Artificial Sequence Synthetic peptide”
PQGIAGQR

SEQ ID NO: 32 - protein, 6 aa, Artificial Sequence
  source: /note=“Description of Artificial Sequence Synthetic peptide”
PLGLAG

SEQ ID NO: 33 - protein, 6 aa, Artificial Sequence
  source: /note=“Description of Artificial Sequence Synthetic peptide”
LALGPR

SEQ ID NO: 34 - protein, 10 aa, Artificial Sequence
  source: /note=“Description of Artificial Sequence Synthetic peptide”
GGPLGMLSQS

SEQ ID NO: 35 - protein, 9 aa, Artificial Sequence
  source: /note=“Description of Artificial Sequence Synthetic peptide”
GGGGRRGGS

SEQ ID NO: 36 - protein, 8 aa, Artificial Sequence
  source: /note=“Description of Artificial Sequence Synthetic peptide”
TGRGPSWV

SEQ ID NO: 37 - protein, 8 aa, Artificial Sequence
  source: /note=“Description of Artificial Sequence Synthetic peptide”
SARGPSRW

SEQ ID NO: 38 - protein, 8 aa, Artificial Sequence
  source: /note=“Description of Artificial Sequence Synthetic peptide”
TARGPSFK

SEQ ID NO: 39 - protein, 7 aa, Artificial Sequence
  source: /note=“Description of Artificial Sequence Synthetic peptide”
TARGPSW

SEQ ID NO: 40 - protein, 8 aa, Artificial Sequence
  source: /note=“Description of Artificial Sequence Synthetic peptide”
GGWHTGRN

SEQ ID NO: 41 - protein, 8 aa, Artificial Sequence
  source: /note=“Description of Artificial Sequence Synthetic peptide”
HTGRSGAL

SEQ ID NO: 42 - protein, 8 aa, Artificial Sequence
  source: /note=“Description of Artificial Sequence Synthetic peptide”
PLTGRSGG

SEQ ID NO: 43 - protein, 7 aa, Artificial Sequence
  source: /note=“Description of Artificial Sequence Synthetic peptide”
LTGRSGA

SEQ ID NO: 44 - protein, 8 aa, Artificial Sequence
  source: /note=“Description of Artificial Sequence Synthetic peptide”
RQARVVNG

SEQ ID NO: 45 - protein, 18 aa, Artificial Sequence
  source: /note=“Description of Artificial Sequence Synthetic peptide”
VHMPLGFLGPRQARVVNG

SEQ ID NO: 46 - protein, 214 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 706 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKRGGGGSGGGGSGGGGSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGQGTLVTVSS

SEQ ID NO: 48 - protein, 706 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGRGTLVTVSSGGGGSGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKR

SEQ ID NO: 49 - protein, 706 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKGGGGSGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSS

SEQ ID NO: 50 - protein, 706 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIK

SEQ ID NO: 51 - protein, 593 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 52 - protein, 601 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 53 - protein, 218 aa, Artificial Sequence
  source: /note=“Description of Artificial Sequence Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 713 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKRGGGGSGGGGSGGGGSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGQGTLVTVSS

SEQ ID NO: 55 - protein, 713 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSQVQLVQSGAELKKPGSSVKVSCKASGYIFTSSWINWVKQAPGQGLEWIGRIDPSDGEVHYNQDFKDKATLTVDKSTNTAYMELSSLRSEDTAVYYCARGFLPWFADWGRGTLVTVSSGGGGSGGGGSGGGGSDIQMTQSPSTLSASVGDRVTITCKASENVDTYVSWYQQKPGKAPKLLIYGASNRYTGVPSRFSGSGSGTDFTLTISSLQPDDFATYYCGQSYNYPFTFGQGTKVEVKR

SEQ ID NO: 56 - protein, 713 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKGGGGSGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSS

SEQ ID NO: 57 - protein, 713 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIK

SEQ ID NO: 58 - protein, 600 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 59 - protein, 608 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 60 - protein, 239 aa, Artificial Sequence
  source: /note=“Description of Artificial Sequence Synthetic polypeptide”
EIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

SEQ ID NO: 61 - protein, 234 aa, Artificial Sequence
  source: /note=“Description of Artificial Sequence Synthetic polypeptide”
EIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIK

SEQ ID NO: 62 - protein, 116 aa, Artificial Sequence [sequence omitted]

SEQ ID NO: 63 - protein, 112 aa, Artificial Sequence
  source: /note=“Description of Artificial Sequence Synthetic polypeptide”
QSVLTQPPSVSGAPGQRVTISCSGSSSNIGSNYVSWYQQLPGTAPKLLIYDNNQRPSGVPDRFSGSKSGTSASLAITGLQSEDEADYYCQSNDASLVEVFGGGTKLTVLGQP

SEQ ID NO: 64 - protein, 119 aa, Artificial Sequence
  source: /note=“Description of Artificial Sequence Synthetic polypeptide”
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARYYSFYTSFDYWGQGTLVTVSS

SEQ ID NO: 65 - protein, 165 aa, Artificial Sequence
  source: /note=“Description of Artificial Sequence Synthetic polypeptide”
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 66 - protein, 165 aa, Artificial Sequence; identical to SEQ ID NO: 65
  source: /note=“Description of Artificial Sequence Synthetic polypeptide”

SEQ ID NO: 67 - protein, 492 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

SEQ ID NO: 68 - protein, 499 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSVHMPLGFLGPRQARVVNAGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

SEQ ID NO: 69 - protein, 487 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIK

SEQ ID NO: 70 - protein, 494 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSVHMPLGFLGPRQARVVNAGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIK

SEQ ID NO: 71 - protein, 407 aa, Artificial Sequence
  source: /note=“Description of Artificial Sequence Synthetic polypeptide”
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 72 - protein, 407 aa, Artificial Sequence; identical to SEQ ID NO: 71
  source: /note=“Description of Artificial Sequence Synthetic polypeptide”

SEQ ID NO: 73 - protein, 492 aa, Artificial Sequence
  source: /note=“Description of Artificial Sequence Synthetic polypeptide”
EIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSSGGGGSGGGGSGPLGVRGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 499 aa, Artificial Sequence
  source: /note=“Description of Artificial Sequence Synthetic polypeptide”
EIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSSGGGGSVHMPLGFLGPRQARVVNAGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 75 - protein, 487 aa, Artificial Sequence
  source: /note=“Description of Artificial Sequence Synthetic polypeptide”
EIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKGGGGSGGGGSGPLGVRGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 76 - protein, 494 aa, Artificial Sequence
  source: /note=“Description of Artificial Sequence Synthetic polypeptide”
EIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIKGGGGSVHMPLGFLGPRQARVVNAGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 77 - protein, 407 aa, Artificial Sequence
  source: /note=“Description of Artificial Sequence Synthetic polypeptide”
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGGGGSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 407 aa, Artificial Sequence; identical to SEQ ID NO: 77
  source: /note=“Description of Artificial Sequence Synthetic polypeptide”

SEQ ID NO: 79 - protein, 705 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

SEQ ID NO: 80 - protein, 694 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

SEQ ID NO: 81 - protein, 700 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIK

SEQ ID NO: 82 - protein, 689 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIK

SEQ ID NO: 83 - protein, 620 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 84 - protein, 620 aa, Artificial Sequence; identical to SEQ ID NO: 83
  source: /note=“Description of Artificial Sequence Synthetic polypeptide”

SEQ ID NO: 85 - protein, 712 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

SEQ ID NO: 86 - protein, 701 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

SEQ ID NO: 87 - protein, 707 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIK

SEQ ID NO: 88 - protein, 696 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSDIQMTQSPSSLSASVGDRVTITCRASQSVSTSSYSYMHWYQQKPGKAPKVLISYASNLESGVPSRFSGSGSGTDFTLTISSLQPEDFATYYCQHSWGIPRTFGQGTKVEIK

SEQ ID NO: 89 - protein, 627 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 90 - protein, 627 aa, Artificial Sequence; identical to SEQ ID NO: 89
  source: /note=“Description of Artificial Sequence Synthetic polypeptide”

SEQ ID NO: 91 - protein, 449 aa, Artificial Sequence
  source: /note=“Description of Artificial Sequence Synthetic polypeptide”
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 214 aa, Artificial Sequence
  source: /note=“Description of Artificial Sequence Synthetic polypeptide”
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 452 aa, Artificial Sequence
  source: /note=“Description of Artificial Sequence Synthetic polypeptide”
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 214 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 450 aa, Artificial Sequence
  source: /note=“Description of Artificial Sequence Synthetic polypeptide”
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 214 aa, Artificial Sequence
  source: /note=“Description of Artificial Sequence Synthetic polypeptide”
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 119 aa, Artificial Sequence
  source: /note=“Description of Artificial Sequence Synthetic polypeptide”
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 98 - protein, 107 aa, Artificial Sequence
  source: /note=“Description of Artificial Sequence Synthetic polypeptide”
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 99 - protein, 122 aa, Artificial Sequence [sequence omitted]

SEQ ID NO: 100 - protein, 107 aa, Artificial Sequence [sequence omitted]

SEQ ID NO: 101 - protein, 120 aa, Artificial Sequence [sequence omitted]

SEQ ID NO: 102 - protein, 107 aa, Artificial Sequence [sequence omitted]

SEQ ID NO: 103 - protein, 120 aa, Artificial Sequence
  source: /note=“Description of Artificial Sequence Synthetic polypeptide”
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 104 - protein, 107 aa, Artificial Sequence
  source: /note=“Description of Artificial Sequence Synthetic polypeptide”
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVL

SEQ ID NO: 105 - protein, 450 aa, Artificial Sequence
  source: /note=“Description of Artificial Sequence Synthetic polypeptide”
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 213 aa, Artificial Sequence
  source: /note=“Description of Artificial Sequence Synthetic polypeptide”
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 107 - protein, 449 aa, Artificial Sequence; identical to SEQ ID NO: 91
  source: /note=“Description of Artificial Sequence Synthetic polypeptide”

SEQ ID NO: 108 - protein, 214 aa, Artificial Sequence; identical to SEQ ID NO: 92
  source: /note=“Description of Artificial Sequence Synthetic polypeptide”

SEQ ID NO: 109 - protein, 452 aa, Artificial Sequence; identical to SEQ ID NO: 93
  source: /note=“Description of Artificial Sequence Synthetic polypeptide”

SEQ ID NO: 110 - protein, 214 aa, Artificial Sequence; identical to SEQ ID NO: 94
  source: /note=“Description of Artificial Sequence Synthetic polypeptide”

SEQ ID NO: 111 - protein, 450 aa, Artificial Sequence; identical to SEQ ID NO: 95
  source: /note=“Description of Artificial Sequence Synthetic polypeptide”

SEQ ID NO: 112 - protein, 214 aa, Artificial Sequence; identical to SEQ ID NO: 96
  source: /note=“Description of Artificial Sequence Synthetic polypeptide”

SEQ ID NO: 113 - protein, 727 aa, Artificial Sequence
  source: /note=“Description of Artificial Sequence Synthetic polypeptide”
MGVKVLFALICIAVAEAQVQLQESGPGLVKPSETLSITCTVSGVSISSYGLHWIRQPPGKSLEWLGVIWASGRTNYNPALKSRVTISGDTSKNQVSLKLSGVTAADTAVYYCAGSNWGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGPLGVRGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

SEQ ID NO: 114 - protein, 723 aa, Artificial Sequence
  source: /note=“Description of Artificial Sequence Synthetic polypeptide”
MGVKVLFALICIAVAEAQVQLQESGPGLVKPSETLSITCTVSGVSISSYGLHWIRQPPGKSLEWLGVIWASGRTNYNPALKSRVTISGDTSKNQVSLKLSGVTAADTAVYYCAGSNWGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSAAGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

SEQ ID NO: 115 - protein, 632 aa, Artificial Sequence
  source: /note=“Description of Artificial Sequence Synthetic polypeptide”
MGVKVLFALICIAVAEAQVQLQESGPGLVKPSETLSITCTVSGVSISSYGLHWIRQPPGKSLEWLGVIWASGRTNYNPALKSRVTISGDTSKNQVSLKLSGVTAADTAVYYCAGSNWGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 116 - protein, 642 aa, Artificial Sequence
  source: /note=“Description of Artificial Sequence Synthetic polypeptide”
MGVKVLFALICIAVAEAQVQLQESGPGLVKPSETLSITCTVSGVSISSYGLHWIRQPPGKSLEWLGVIWASGRTNYNPALKSRVTISGDTSKNQVSLKLSGVTAADTAVYYCAGSNWGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 117 - protein, 632 aa, Artificial Sequence
  source: /note=“Description of Artificial Sequence Synthetic polypeptide”
MGVKVLFALICIAVAEAQVQLQESGPGLVKPSETLSITCTVSGVSISSYGLHWIRQPPGKSLEWLGVIWASGRTNYNPALKSRVTISGDTSKNQVSLKLSGVTAADTAVYYCAGSNWGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 118 - protein, 642 aa, Artificial Sequence
  source: /note=“Description of Artificial Sequence Synthetic polypeptide”
MGVKVLFALICIAVAEAQVQLQESGPGLVKPSETLSITCTVSGVSISSYGLHWIRQPPGKSLEWLGVIWASGRTNYNPALKSRVTISGDTSKNQVSLKLSGVTAADTAVYYCAGSNWGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGGGGSGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 119 - protein, 641 aa, Artificial Sequence
  source: /note=“Description of Artificial Sequence Synthetic polypeptide”
MGVKVLFALICIAVAEAQVQLQESGPGLVKPSETLSITCTVSGVSISSYGLHWIRQPPGKSLEWLGVIWASGRTNYNPALKSRVTISGDTSKNQVSLKLSGVTAADTAVYYCAGSNWGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNRFTQKSLSLSLGAGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 120 - protein, 236 aa, Artificial Sequence
  source: /note=“Description of Artificial Sequence Synthetic polypeptide”
MDMRVPAQLLGLLLLWLPGAKCAIRMTQSPSSFSASAGDRVTITCRASENIYSYLAWYQQKPGKSPKLLIYNAKTLAEGVPSRFSGSGSGTDFTLTISSLQPEDFATYYCQHHYAIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 461 aa, Artificial Sequence
  source: /note=“Description of Artificial Sequence Synthetic polypeptide”
MGVKVLFALICIAVAEAQVQLQESGPGLVKPSETLSITCTVSGVSISSYGLHWIRQPPGKSLEWLGVIWASGRTNYNPALKSRVTISGDTSKNQVSLKLSGVTAADTAVYYCAGSNWGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 122 - protein, 5 aa, Artificial Sequence
  source: /note=“Description of Artificial Sequence Synthetic peptide”
GGGGS

SEQ ID NO: 123 - protein, 10 aa, Artificial Sequence
  source: /note=“Description of Artificial Sequence Synthetic peptide”
GGGGSGGGGS

SEQ ID NO: 124 - protein, 15 aa, Artificial Sequence
  source: /note=“Description of Artificial Sequence Synthetic peptide”
GGGGSGGGGSGGGGS

SEQ ID NO: 125 - protein, 22 aa, Artificial Sequence
  source: /note=“Description of Artificial Sequence Synthetic peptide”
GGGGSGGGGSAAGGGGSGGGGS

SEQ ID NO: 126 - protein, 895 aa, Artificial Sequence
  source: /note=“Description of Artificial Sequence Synthetic polypeptide”
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGPLGLAGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

SEQ ID NO: 127 - protein, 893 aa, Artificial Sequence
  source: /note=“Description of Artificial Sequence Synthetic polypeptide”
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKEGGGGSGGGGSGPLGLAGGGGSGGGGSEIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKGGGGSGGGGSGGGGSQVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSS

(19) United States
(12) Patent Application Publication
(10) Pub. No.: US 2024/0076355 A1
(43) Pub. Date: Mar. 7, 2024

(54) Interferon Prodrugs and Methods of Making and Using the Same

(71) Applicant: ASKGENE PHARMA, INC., Camarillo, CA (US)

(72) Inventor: Yuefeng LU, Moorpark, CA (US)

(73) Assignee: ASKGENE PHARMA, INC., Camarillo, CA (US)

(21) Appl. No.: 18/259,695

(22) PCT Filed: Jan. 14, 2022
(86) PCT No.: PCT/US2022/012629

§ 371 Date: Jun. 28, 2023

Related U.S. Application Data
(60) Provisional application No. 63/137,370, filed on Jan. 14, 2021.

(51) Int. Cl. C07K 14/715; A61K 47/68; A61P 35/00; C07K 16/24; C07K 16/28

(52) U.S. Cl. CPC C07K 14/7156; A61K 47/6813; A61K 47/6845; A61K 47/6849; A61K 47/6889; A61P 35/00; C07K 16/249; C07K 16/2827

(57) ABSTRACT

Provided herein are interferon prodrugs and methods of making and using thereof for stimulating the immune system, or treating cancer or an infectious disease.

### CROSS REFERENCE TO RELATED APPLICATION

The present application claims priority from U.S. Provisional Application No. 63/137,370, filed on Jan. 14, 2021, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jan. 13, 2022, is named 025471_WO0014 SL.txt and is 397,253 bytes in size.

### BACKGROUND

Interferons (IFN) modulate a myriad of immune functions, including regulating and activating an immune response in response to viral infections. There are several types of INFs based on the type of receptor through which they signal. Type I IFNs bind to a cell surface receptor complex known as the IFN-α/β receptor (IFNAR), which consists of IFNAR1 and IFNAR2 chains (Weerd et al. The Journal of Biological Chemistry (2007) 282 (28): 20053-7). Type II IFNs (IFNγ in humans) are released by cytotoxic and type-1 helper T cells and binds to IFNγ receptor (IFNGR), which consists of IFNGR1 and IFNGR2 chains (Parkin and Cohen. Immunology (2001) 357(9270):1777-89).

Interferon-γ (IFNγ) and Interferon-α (IFNα) play important roles in regulating the immune system. They have been tested in clinical trials as treatment for cancer. For example, IFNγ was tested to treat ovarian cancer (Marth et al., Int. J. Gynecol. (Cancer) (2006) 16:1522-1528). It was found effective in the Phase 2 trial using dosages of up to 0.1 mg. While the dosage was low, there were significant side effects. IFNα-2b was approved as a therapeutic protein that can be used to treat some types of cancer, either in monotherapy or combination therapy with other anticancer drugs. Yet the flu-like syndrome associated with its use limited its clinical application (Kirwood., Semin Oncol (2002) 29(3 Suppl 7):18-26).

There is a need to develop IFNγ and IFNα-based cancer therapeutics which are more tumor site-selective, has less severe side effects, and/or has improved efficacy. The disclosures of all publications, patents, and patent applications referred to herein are each hereby incorporated herein by reference in their entireties.

### SUMMARY OF THE INVENTION

The present disclosure provides a prodrug comprising a human interferon (IFN) agonist polypeptide, a masking moiety, and a carrier moiety, wherein the masking moiety comprises an antigen-binding fragment of an antibody that binds to the human interferon agonist polypeptide and inhibits a biological activity of the human interferon agonist polypeptide, the human interferon agonist polypeptide is interferon alpha (IFNα) and is fused to the carrier moiety, and the masking moiety is fused to the human interferon agonist polypeptide or to the carrier moiety, optionally through a peptide linker.

In one aspect, the present disclosure provides a prodrug comprising a human interferon (IFN) agonist polypeptide, a masking moiety, and a carrier moiety, wherein the masking moiety binds to the human interferon agonist polypeptide and inhibits a biological activity of the human interferon agonist polypeptide, the human interferon agonist polypeptide is fused to the carrier moiety, and the masking moiety is fused to the human interferon agonist polypeptide or to the carrier moiety, optionally through a peptide linker.

In some embodiments, the IFNα is IFNα-2a, IFNα-2b, or an analog thereof. In some embodiments, the IFN is IFNα-2a, IFNα-2b, IFNγ, or an analog thereof.

In particular embodiments, the INFα-2a comprises an amino acid sequence selected from SEQ ID NO: 65 or at least 95% identical to SEQ ID NO: 65.

In particular embodiments, the IFNγ comprises an amino acid sequence selected from SEQ ID NO: 5, 6, and 7, or at least 90% identical to SEQ ID NO: 5, 6, or 7. In particular embodiments, the INFα-2b comprises an amino acid sequence selected from SEQ ID NO: 66 or at least 95% identical to SEQ ID NO: 66.

In some embodiments, the IFN agonist polypeptide is fused to the carrier through a non-cleavable peptide linker and the masking moiety is fused to the carrier through a cleavable peptide linker or a non-cleavable peptide linker. In other embodiments, the IFN agonist polypeptide is fused to the carrier through a non-cleavable peptide linker or a cleavable linker and the masking moiety is fused to the carrier through a non-cleavable peptide linker.

In some embodiments, the carrier comprises an antibody Fc domain with a first Fc polypeptide chain with knob mutations and a second Fc polypeptide chain with hole mutations; wherein the IFN agonist polypeptide is fused to the first the carrier through a cleavable peptide linker and the masking moiety is fused to the second Fc polypeptide chain through a non-cleavable peptide linker.

In some embodiments, the carrier moiety is an antibody Fc domain or an antibody comprising knobs-into-holes mutations, and wherein the human IFN agonist polypeptide and its masking moiety are fused to different polypeptide chains of the antibody Fc domain, or to the different heavy chains of the antibody.

In some embodiments, the carrier is an antibody and the prodrug comprises two IFN agonist polypeptides fused to the C-terminus of the two heavy chains of the antibody through non-cleavable peptide linkers and two masking moieties that are fused to the two IFN agonist polypeptide through cleavable peptide linkers.

In some embodiments, the peptide linker is a cleavable peptide linker comprising a substrate sequence of urokinase-type plasminogen activator (uPA), matrix metallopeptidase (MT1-MMP), matrix metallopeptidase 2 (MMP2), MMP9, matriptase, legumain, plasmin, TMPRSS-3/4, cathepsin, caspase, human neutrophil elastase, beta-secretase, or PSA, or (i) both uPA and MMP2, (ii) both uPA and MMP9, or (iii) matriptase, MMP2 and MMP9. In particular embodiments, the cleavable peptide linker comprises an amino acid sequence selected from SEQ ID NOs: 26-45. In other particular embodiments, the non-cleavable peptide linker comprises an amino acid sequence selected from SEQ ID NOs: 122-125. In some embodiments, the cleavable peptide linker is cleavable by one or more proteases located at a tumor site or its surrounding environment, and the cleavage leads to activation of the prodrug at the tumor site or surrounding environment.

In some embodiments, the masking moiety inhibits the binding of the IFN agonist polypeptide to an IFN receptor. In particular embodiments, the masking moiety comprises a single chain fragment variable (scFv) comprising a heavy chain variable domain with an amino acid sequence as shown SEQ ID NO: 1 and a light chain variable domain with an amino acid sequence as shown in SEQ ID NO: 2, or a heavy chain variable domain with an amino acid sequence as shown SEQ ID NO: 3 and a light chain variable domain with an amino acid sequence as shown in SEQ ID NO: 4.

In some embodiments, the masking moiety is selected from interferon gamma receptor 1 extracellular domain (IFNGR1-ECD) or a functional analog thereof, or an antibody or a binding fragment thereof which binds to IFNγ. In particular embodiments, the masking moiety comprises IFNGR1-ECD or a functional analog thereof, and optionally wherein the IFNGR1-ECD comprises an amino acid sequence selected from SEQ ID NOs: 8 and 9.

In some embodiments, the carrier moiety is an antibody Fc domain, an antibody, or an antigen-binding fragment of an antibody. In some embodiments, the carrier moiety comprises an antibody or antigen-binding fragment thereof that binds to an antigen expressed on the surface of a tumor cell, a cell in the tumor microenvironment, a cancer cell, or an immune cell. In some embodiments, the immune cell is selected from an NK cell, a T cell, a B cell, and a macrophage.

In some embodiments, the carrier moiety comprises an antibody or antigen-binding fragment thereof that binds to an antigen selected from PD-1, LAG-3, TIGIT, SIRPα, ILT2, CD206, NKD2G, CTLA-4, CD8, NKG2A, CD16a, CD38, BCMA, cell-surface glycoprotein CD2 subset 1 (CS1), PD-L1, CD47, CMET, EGFR, ROR1, TROP-2, HER2, CLDN18.2, and VEGFR2.

In particular embodiments, In other aspects, the present disclosure provides also a prodrug comprising a pharmaceutical composition and a pharmaceutically acceptable excipient; a polynucleotide or polynucleotides encoding the prodrug; an expression vector or vectors comprising the polynucleotide or polynucleotides; and a host cell comprising the vector(s), wherein the host cell may be a prokaryotic cell or an eukaryotic cell such as a mammalian cell. In some embodiments, the mammalian host cell has the gene or genes encoding uPA, MMP-2 and/or MMP-9 knocked out (e.g., containing null mutations of one or more of these genes).

In some embodiments, the present disclosure also provides a method of making the prodrug, comprising culturing the host cell under conditions that allow expression of the prodrug, wherein the host cell is a mammalian cell, and isolating the prodrug.

The present disclosure also provides a method of treating a cancer or an infectious disease or stimulating the immune system in a patient in need thereof, comprising administering to the patient a therapeutically effective amount of the prodrug or pharmaceutical composition of the present disclosure. The patient may have, for example, a viral infection or a cancer, for example a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer.

Also provided herein are a prodrugs or pharmaceutical compositions for use in treating a cancer or an infectious disease or stimulating the immune system in a patient in need thereof; use of a prodrug for the manufacture of a medicament for treating a cancer or an infectious disease or stimulating the immune system in a patient in need thereof; and articles of manufacture (e.g., kits) comprising one or more dosing units of the present prodrug.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIGS. 1A, 1B, and 1C show the schematic structures of interferon prodrugs. FIG. 1A shows an activatable IFNα prodrug with an antibody as its carrier and a cleavable linker, wherein the prodrug comprises two identical light chains, a heavy chain-mask fusion polypeptide chain comprising a cleavable linker, and a heavy chain-cytokine fusion polypeptide chain. FIG. 1B shows an IFNα prodrug with an antibody as its carrier, wherein the prodrug comprises two identical light chains, a heavy chain-mask fusion polypeptide chain comprising a non-cleavable linker, and a heavy chain-cytokine fusion polypeptide chain. FIG. 1C shows an activatable IFNα prodrug with an antibody as its carrier and cleavable linkers, wherein the prodrug comprises two identical light chains and two identical heavy chain polypeptide chains; wherein each of the heavy chain polypeptide chains comprises a cytokine fused to the C-terminus of the heavy chain and a mask fused to the C-terminus of the cytokine through a cleavable peptide linker.

FIG. 2 shows the SEC-HPLC data of the 5T4 antibody JR11.60.1 and prodrugs JR11.60.2 and JR11.60.3 after Protein A affinity chromatography purification. The structure of prodrugs JR11.60.2 and JR11.60.3 is shown in FIG. 1A.

FIG. 3A shows the non-reduced and reduced SDS-PAGE analysis of JR11.60.1, JR11.60.2 and JR11.60.3 after Protein A affinity chromatography purification. FIG. 3B shows the SDS-PAGE analysis of the activatable fusion molecules prior to and after activation under non-reduced conditions.

FIG. 4 shows the proliferation of Daudi cells at various concentrations of 5T4 antibody-IFNα prodrug molecules JR11.60.2 and JR11.60.3 as well as activated prodrug molecule JR11.60.2. Interferon alpha-2b is also included as the positive control.

### DETAILED DESCRIPTION OF THE INVENTION

As used herein and in the appended claims, the singular forms “a,” “or,” and “the” include plural referents unless the context clearly dictates otherwise. Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “antigen-binding moiety” refers to a polypeptide or a set of interacting polypeptides that specifically bind to an antigen, and includes, but is not limited to, an antibody (e.g., a monoclonal antibody, polyclonal antibody, a multi-specific antibody, a dual specific or bispecific antibody, an anti-idiotypic antibody, or a bifunctional hybrid antibody) or an antigen-binding fragment thereof (e.g., a Fab, a Fab′, a F(ab′)₂, a Fv, a disulfide linked Fv, a scFv, a single domain antibody (dAb), or a diabody), a single chain antibody, and an Fc-containing polypeptide such as an immunoadhesin. In some embodiments, the antibody may be of any heavy chain isotype (e.g., IgG, IgA, IgM, IgE, or IgD) or subtype (e.g., IgG₁, IgG₂, IgG₃, or IgG₄). In some embodiments, the antibody may be of any light chain isotype (e.g., kappa or lambda). The antibody may be human, non-human (e.g., from mouse, rat, rabbit, goat, or another non-human animal), chimeric (e.g., with a non-human variable region and a human constant region), or humanized (e.g., with non-human CDRs and human framework and constant regions). In some embodiments, the antibody is a derivatized antibody.

The terms “cytokine agonist polypeptide” or “cytokine moiety” refers to a wildtype cytokine, or an analog thereof. An analog of a wildtype cytokine has the same biological specificity (e.g., binding to the same receptor(s) and activating the same target cells) as the wildtype cytokine, although the activity level of the analog may be different from that of the wildtype cytokine. The analog may be, for example, a mutein (i.e., mutated polypeptide) of the wildtype cytokine, and may comprise at least one, at least two, at least three, at least four, at least five, at least six, at least seven, at least eight, at least nine, or at least ten mutations relative to the wildtype cytokine.

The term “cytokine mask” or “masking moiety” refers to a moiety (e.g., a polypeptide) that binds to a cytokine, thereby inhibiting the cytokine from binding to its receptor on the surface of a target cell and/or exerting its biological functions while being bound by the mask. Examples of a cytokine mask include, without limitations, a polypeptide derived from an extracellular domain of the cytokine's natural receptor that makes contact with the cytokine.

The term “effective amount” or “therapeutically effective amount” refers to an amount of a compound or composition sufficient to treat a specified disorder, condition, or disease, such as ameliorate, palliate, lessen, and/or delay one or more of its symptoms.

The term “functional analog” refers to a molecule that has the same biological specificity (e.g., binding to the same ligand) and/or activity (e.g., activating or inhibiting a target cell) as a reference molecule.

The term “fused” or “fusion” in reference to two polypeptide sequences refers to the joining of the two polypeptide sequences through a backbone peptide bond. Two polypeptides may be fused directly or through a peptide linker that is one or more amino acids long. A fusion polypeptide may be made by recombinant technology from a coding sequence containing the respective coding sequences for the two fusion partners, with or without a coding sequence for a peptide linker in between. In some embodiments, fusion encompasses chemical conjugation.

The term “pharmaceutically acceptable excipient” when used to refer to an ingredient in a composition means that the excipient is suitable for administration to a treatment subject, including a human subject, without undue deleterious side effects to the subject and without affecting the biological activity of the active pharmaceutical ingredient (API).

The term “subject” refers to a mammal and includes, but is not limited to, a human, a pet (e.g., a canine or a feline), a farm animal (e.g., cattle or horse), a rodent, or a primate.

As used herein, “treatment” or “treating” is an approach for obtaining beneficial or desired clinical results. Beneficial or desired clinical results include, but are not limited to, one or more of the following: alleviating one or more symptoms resulting from a disease, diminishing the extent of a disease, ameliorating a disease state, stabilizing a disease (e.g., preventing or delaying the worsening or progression of the disease), preventing or delaying the spread (e.g., metastasis) of a disease, preventing or delaying the recurrence of a disease, providing partial or total remission of a disease, decreasing the dose of one or more other medications required to treat a disease, increasing the patient's quality of life, and/or prolonging survival. The methods of the present disclosure contemplate any one or more of these aspects of treatment.

It is to be understood that one, some or all of the properties of the various embodiments described herein may be combined to form other embodiments of the present invention. The section headings used herein are for organizational purposes only and are not to be construed as limiting the subject matter described thereunder.

### I. IFN Prodrugs

Described herein are novel cytokine prodrugs which comprise an interferon (IFN) agonist molecule (cytokine moiety). In some embodiments, the prodrugs are activatable at the site of a tumor.

The IFN prodrugs have fewer side effects, better in vivo PK profiles (e.g., longer half-life) and better target specificity, and are more efficacious as compared to prior IFN therapeutics. The present prodrugs comprise an IFN agonist molecule, a masking moiety, a carrier, and an optional peptide linker. In some embodiments, the peptide linker is a cleavable or a non-cleavable peptide linker.

The interferon agonist molecule disclosed here may be selected from IFNγ, IFNα-2b, and IFNα-2a. In some embodiments, IFNγ comprises an amino acid as shown in SEQ ID NO: 5, 6, or 7. In some embodiments, an IFNγ analog comprises an amino acid that is at least 90% identical to SEQ ID NO: 5, 6 or 7. In other embodiments, an IFNα-2a agonist polypeptide comprises an amino acid sequence of SEQ ID NO: 65 or at least 90% identical to SEQ ID NO: 65. In other embodiments, IFNα-2b agonist polypeptide comprises an amino acid sequence of SEQ ID NO: 66 or at least 90% identical to SEQ ID NO: 66.

In some embodiments, the IFN prodrugs comprise at least one masking moiety. The masking moieties may be linked to the cytokine moiety or to the carrier moiety through a peptide linker. In some embodiments, the peptide linker is a non-cleavable peptide linker. In some embodiments, the peptide linker is a cleavable peptide linker. In some embodiments, the cleavable peptide linker comprises one or more cleavable moieties, which are substrates of proteases typically found at a tumor site. The mask inhibits the cytokine moiety's biological functions while the mask is binding to it. In specific embodiments, the mask inhibits a biological activity of IFNγ, IFNα-2b, or IFNα-2a or an analog thereof. The prodrugs may be activated at a target site (e.g., at a tumor site or the surrounding environment) in the patient by cleavage of the linker and the consequent release of the cytokine mask from the prodrug, exposing the previously masked cytokine moiety and allowing the cytokine moiety to bind to its receptor on a target cell and exert its biological functions on the target cell.

In some embodiments, the carriers for the prodrugs are antigen-binding moieties that bind an antigen at a target site (e.g., tumor surface). In some embodiments, the present prodrugs are metabolized to become active in the body at a target site targeted by the carrier. In further embodiments, the carrier in the prodrug is an antibody targeting a tumor antigen such that the prodrug is delivered to a tumor site in a patient and is metabolized locally (e.g., inside or in the vicinity of the tumor microenvironment). This occurs following cleavage of a linker linking the cytokine mask to the carrier or the cytokine moiety, which makes the cytokine moiety available to interact with its receptor on a target cell and stimulate the target immune cells locally.

In some embodiments, the carrier is selected from an albumin, a Fc fragment, an Fc domain, a polyethylene glycol (PEG), or an antibody or antigen-binding fragment thereof.

A. Masking Moieties of the IFN Prodrugs

In some embodiments, the masking moiety comprises an IFNγ receptor 1 extracellular domain (IFNGR1-ECD) or a fragment thereof. In some embodiment, the masking moiety comprises a scFv or Fab with specificity to IFNγ, IFNα-2b and/or IFNα-2a. In some embodiments, the masking moiety inhibits a biological activity of IFNγ, IFNα-2b or IFNα-2a or its analog. In some embodiments, the masking moiety comprises a scFv, wherein the scFv has an amino acid sequence at least 99% identical to SEQ ID NOs: 1-4. In some embodiments, the masking moiety comprises a scFv, wherein the scFv has an amino acid sequence at least 99% identical to SEQ ID NOs: 60 and 61. In some embodiments, the scFv or Fab comprises the same light chain CDRs and heavy chain CDRs as the antibody rontalizumab. In some embodiments, the scFv or Fab comprises a VL domain with an amino acid sequence of SEQ ID NO: 62 or at least 90% identical to SEQ ID NO: 62. In some embodiments, the scFv or Fab comprises a VL domain with an amino acid sequence of SEQ ID NO: 63 or at least 90% identical to SEQ ID NO: 63 and a VH domain with an amino acid sequence of SEQ ID NO: 64 or at least 90% identical to SEQ ID NO: 64. In some embodiments, the scFv or Fab comprises the same light chain CDRs and heavy chain CDRs as an IFNα antibody disclosed in patent application WO 2016/112497A1. In some embodiments, the scFv comprises the same heavy chain CDR1, CDR2, CDR3, and light chain CDR1, CDR2, CDR3 as sifalimumab or rontalizumab.

B. Carrier Moieties of the Prodrugs

The carrier moieties of the present prodrugs may be an antigen-binding moiety, or a moiety that is not antigen-binding. The carrier moiety may improve the PK profiles, such as serum half-life, of the cytokine agonist polypeptide and may also target the cytokine agonist polypeptide to a target site in the body, such as a tumor site.

1. Non-Antigen-Binding Carrier Moieties

Non-antigen-binding carrier moieties may be used for the present prodrugs. For example, an antibody Fc domain (e.g., a human IgG₁, IgG₂, IgG₃, or IgG₄ Fc), a polymer (e.g., PEG), an albumin (e.g., a human albumin) or a fragment thereof, or a nanoparticle can be used.

The carrier moiety of the prodrug may comprise an albumin (e.g., human serum albumin) or a fragment thereof. In some embodiments, the albumin or albumin fragment is about 85% or more, about 90% or more, about 91% or more, about 92% or more, about 93% or more, about 94% or more, about 95% or more, about 96% or more, about 97% or more, about 98% or more, about 99% or more, about 99.5% or more, or about 99.8% or more identical to human serum albumin or a fragment thereof.

In some embodiments, the carrier moiety comprises an albumin fragment (e.g., a human serum albumin fragment) that is about 10 or more, 20 or more, 30 or more 40 or more, 50 or more, 60 or more, 70 or more, 80 or more, 90 or more, 100 or more, 120 or more, 140 or more, 160 or more, 180 or more, 200 or more, 250 or more, 300 or more, 350 or more, 400 or more, 450 or more, 500 or more, or 550 or more amino acids in length. In some embodiments, the albumin fragment is between about 10 amino acids and about 584 amino acids in length (such as between about 10 and about 20, about 20 and about 40, about 40 and about 80, about 80 and about 160, about 160 and about 250, about 250 and about 350, about 350 and about 450, or about 450 and about 550 amino acids in length). In some embodiments, the albumin fragment includes the Sudlow I domain or a fragment thereof, or the Sudlow II domain or the fragment.

In some embodiments, the carrier is an antibody Fc fragment. Fc is a dimeric molecule that has two N-terminals and two C-terminals. In some embodiments, the cytokine moiety can be fused to one Fc polypeptide chain in a dimeric Fc fragment, and the masking moieties can be fused to the other Fc polypeptide chain. In some embodiments, both the cytokine moiety and the masking moiety are fused to the C-terminal of each polypeptide chain of the dimeric Fc fragment. In some embodiments, both the cytokine moiety and the masking moieties are fused to the N-terminal of each polypeptide chain of the dimeric Fc fragment. In some embodiments, two cytokine moieties are fused to the C-terminal of each heavy chain of the dimeric Fc fragment and two masking moieties are fused to the C-terminal of each cytokine moiety.

2. Antigen-Binding (Carrier) Moieties

The antigen-binding moiety may be an antibody or an antigen-binding fragment thereof, or an immunoadhesin, or a ligand of a receptor. In some embodiments, the antigen-binding moiety is a full-length antibody with two heavy chains and two light chains, a Fab fragment, a Fab′ fragment, a F(ab′)₂ fragment, a Fv fragment, a disulfide linked Fv fragment, a single domain antibody, a nanobody, or a single-chain variable fragment (scFv). In some embodiments, the antigen-binding moiety is a bispecific antigen-binding moiety and can bind to two different antigens or two different epitopes on the same antigen. The antigen-binding moiety may provide additional and potentially synergetic therapeutic efficacy to the cytokine agonist polypeptide. In some embodiments, the antigen-binding moiety comprises a full-length antibody heavy chain or a full-length antibody light chain. In some embodiments, the antigen-binding moiety includes an antibody heavy chain fragment or an antibody light chain fragment.

In some embodiments, the cytokine moiety is fused to the C-terminus of one of the heavy chains of an antibody, and the cytokine's mask is fused to the C-terminus of the other heavy chain of the antibody through a peptide linker (optionally a cleavable linker), wherein the two heavy chains optionally contain mutations that allow the specific pairing of the two different heavy chains.

Strategies of forming heterodimers for Fc-fusion polypeptides or bispecific antibodies are well known (see, e.g., Spies et al., Mol Imm. (2015) 67(2)(A):95-106). For example, the two heavy chain polypeptides in the prodrug may form stable heterodimers through “knobs-into-holes” mutations. “Knobs-into-holes” mutations are made to promote the formation of the heterodimers of the antibody heavy chains and are commonly used to make bispecific antibodies (see, e.g., U.S. Pat. No. 8,642,745). For example, the Fc domain of the antibody may comprise a T366W mutation in the CH3 domain of the “knob chain” and T366S, L368A, and/or Y407V mutations in the CH3 domain of the “hole chain.” An additional interchain disulfide bridge between the CH3 domains can also be used, e.g., by introducing a Y349C mutation into the CH3 domain of the “knobs chain” and an E356C or S354C mutation into the CH3 domain of the “hole chain” (see, e.g., Merchant et al., Nature Biotech (1998) 16:677-81). In other embodiments, the antibody moiety may comprise Y349C and/or T366W mutations in one of the two CH3 domains, and E356C, T366S, L368A, and/or Y407V mutations in the other CH3 domain. In certain embodiments, the antibody moiety may comprise Y349C and/or T366W mutations in one of the two CH3 domains, and S354C (or E356C), T366S, L368A, and/or Y407V mutations in the other CH3 domain, with the additional Y349C mutation in one CH3 domain and the additional E356C or S354C mutation in the other CH3 domain, forming an interchain disulfide bridge (numbering always according to EU index of Kabat; Kabat et al., “Sequences of Proteins of Immunological Interest,” 5th ed., Public Health Service, National Institutes of Health, Bethesda, Md. (1991)). Other knobs-into-holes technologies, such as those described in EP1870459A1, can be used alternatively or additionally. Thus, another example of knobs-into-holes mutations for an antibody moiety is having R409D/K370E mutations in the CH3 domain of the “knob chain” and D399K/E357K mutations in the CH3 domain of the “hole chain” (EU numbering).

In some embodiments, the antibody moiety in the prodrug comprises L234A and L235A (“LALA”) mutations in its Fc domain. The LALA mutations eliminate complement binding and fixation as well as Fcγ dependent ADCC (see, e.g., Hezareh et al. J. Virol. (2001) 75(24):12161-8). In further embodiments, the LALA mutations are present in the antibody moiety in addition to the knobs-into-holes mutations.

In some embodiments, the antibody moiety comprises the M252Y/S254T/T256E (“YTE”) mutations in the Fc domain. The YTE mutations allow the simultaneous modulation of serum half-life, tissue distribution and activity of IgG₁ (see Dall'Acqua et al., J Blot Chem. (2006) 281: 23514-24; and Robbie et al., Antimicrob Agents Chemother. (2013) 57(12):6147-53). In further embodiments, the YTE mutations are present in the antibody moiety in addition to the knobs-into-holes mutations. In particular embodiments, the antibody moiety has YTE, LALA and knobs-into-holes mutations or any combination thereof.

In some embodiments, the antibody binds to PD-L1. In some embodiments, the antibody binds to CEA. In some embodiments, the antibody binds to an antigen on a tumor cell, for examples, CD38, BCMA, 5T4, FAP, Trop-2, PD-L1, HER-2, EGFR, Claudin 18.2, DLL-3, GCP3, and CEA. The antibody may or may not have Antibody-Dependent Cellular Cytotoxicity (ADCC) activity. The antibody may also be further conjugated with cytotoxic drugs. In some embodiments, the antibody binds to a target on the surface of an immune cell and has the ability to activate the immune cell and enhance its anti-cancer activity. Examples of such antibodies include any one of the following: an ILT-2 antibody, a PD-1 antibody, a LAG3 antibody, a TIGIT antibody, a TGF-beta antibody, or a CTLA4 antibody.

The antigen-binding moiety (carrier moiety) can bind an antigen on the surface of a cell, such as a cancer cell. In some embodiments, the antigen-binding moiety is a bispecific antigen-binging moiety that can bind to two different antigens or two different epitopes on the same antigen. In some embodiments, the antigen-binding moiety binds to Guanyl cyclase C (GCC), carbohydrate antigen 19-9 (CA19-9), glycoprotein A33 (gpA33), mucin 1 (MUC1), carcinoembryonic antigen (CEA), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1) 1, vascular endothelial growth factor receptor 2 (VEGFR2), Nectin-4, Liv-1, glycoprotein NMB (GPNMB), prostate-specific membrane antigen (PSMA), Trop-2, carbonic anhydrase IX (CA9), endothelin B receptor (ETBR), Thomsen-Friedenrech antigen (TF), sodium-dependent phosphate transport protein 2B (NaPi2b), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD30, CD33, CD38, BCMA, CD40, CD56, CD66e, CD70, CD74, CD79b, CD98, CD123, CD138, CD352, programmed death ligand 1 (PD-L1), Claudin 18.2, Claudin 6, PSMA, or FAP-alpha. In some embodiments, the antigen-binding moiety binds to an epidermal growth factor (EGF)-like domain of DLL3. In some embodiments, the antigen-binding moiety binds to a Delta/Serrate/Lag2 (DSL)-like domain of DLL3. In some embodiments, the antigen-binding moiety binds to an epitope located after the 374^{th} amino acid of GPC3. In some embodiments, the antigen-binding moiety binds to a heparin sulfate glycan of GPC3. In some embodiments, the antigen-binding moiety binds to Claudin 18.2 and does not bind to Claudin 18.1. In some embodiments, the antigen-binding moiety binds to Claudin 18.1 with at least 10 times weaker binding affinity than to Claudin 18.2.

The antigen-binding moiety can bind an antigen on the surface of a cell, such as an immune cell, for example T cells, NK cells, B cells, and macrophages. In some embodiments, the antigen-binding moiety is a bispecific antigen-binging moiety that can bind to two different antigens or two different epitopes on the same antigen. In some embodiments, the antigen-binding moiety binds to ILT2, PD-1, LAG-3, TIM-3, CTLA-4, or TGF-beta.

In some embodiments, the antigen-binding moiety includes an antibody or fragment thereof known in the art that binds to PD-1 and disrupts the interaction between the PD-1 and its ligand (PD-L1) to stimulate an anti-tumor immune response. In some embodiments, the antibody or antigen-binding portion thereof binds specifically to PD-1. For example, antibodies that target PD-1 and which can find use in the present invention include, but are not limited to, nivolumab (BMS-936558, Bristol-Myers Squibb), pembrolizumab (lambrolizumab, MK03475 or MK-3475, Merck), humanized anti-PD-1 antibody JS001 (ShangHai JunShi), monoclonal anti-PD-1 antibody TSR-042 (Tesaro, Inc.), pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)—BioXcell cat #BP0146. Other suitable anti-PD-1 antibodies include those disclosed in U.S. Pat. No. 8,008,449. In some embodiments, the antibody or antigen-binding portion thereof binds specifically to PD-L1 and inhibits its interaction with PD-1, thereby increasing immune activity. Any antibodies known in the art which bind to PD-L1 and disrupt the interaction between PD-1 and PD-L1, and stimulates an anti-tumor immune response, are suitable for use in combination treatment methods disclosed herein. As an example, antibodies that target PD-L1 include BMS-936559 (Bristol-Myers Squibb) and MPDL3280A (Genentech; currently in human trials). Other suitable antibodies that target PD-L1 are disclosed in U.S. Pat. No. 7,943,743. It will be understood by one of ordinary skill that any antibody which binds to PD-1 or PD-L1, disrupts the PD-1/PD-L1 interaction, and stimulates an anti-tumor immune response, is suitable for use in the combination treatment methods disclosed herein.

In some embodiments, the carrier is an antibody against human PD-L1 is selected from ASKB1296, avelumab, atezolizumab and durvalumab.

In some embodiments, the carrier is an antibody against human CD38 is selected from daratumumab, isatuximab, and felzartamab. In some embodiments, the carrier is elotuzumab. In some embodiments, the carrier moiety comprises an antibody comprising: i) a heavy chain variable domain with an amino acid sequence of SEQ ID NO: 97 or at least 95% identical to SEQ ID NO: 97, and a light chain variable domain with an amino acid sequence of SEQ ID NO: 98 or at least 95% identical to SEQ ID NO: 98; or (ii) a heavy chain variable domain with an amino acid sequence of SEQ ID NO: 99 or at least 95% identical to SEQ ID NO: 99, and a light chain variable domain with an amino acid sequence of SEQ ID NO: 100 or at least 95% identical to SEQ ID NO: 100; (iii) a heavy chain variable domain with an amino acid sequence of SEQ ID NO: 101 or at least 95% identical to SEQ ID NO: 101, and a light chain variable domain with an amino acid sequence of SEQ ID NO: 102 or at least 95% identical to SEQ ID NO: 102; or (iv) a heavy chain variable domain with an amino acid sequence of SEQ ID NO: 103 or at least 95% identical to SEQ ID NO: 103, and a light chain variable domain with an amino acid sequence of SEQ ID NO: 104 or at least 95% identical to SEQ ID NO: 104.

Exemplary antigen-binding moieties include daratumumab, isatuximab, and felzartamab, trastuzumab, rituximab, brentuximab, cetuximab, panitumumab, GC33 (or a humanized version thereof), anti-EGFR antibody mAb806 (or a humanized version thereof), anti-FAP-alpha antibody sibrotuzumab (BIBH1), and fragments thereof. In some embodiments, the antigen-binding moiety that has at least 90%, 91%, 92%, 93%, 94%, 95%, 96%, 97%, 98%, or 99% identity to daratumumab, isatuximab, and felzartama, trastuzumab, rituximab, brentuximab, cetuximab, or panitumumab, GC33 (or a humanized version thereof), anti-EGFR antibody mAb806 (or a humanized version thereof), sibrotuzumab (BIBH1), or a fragment thereof. In some embodiments, the antigen-binding moiety has an antibody heavy chain with at least 90%, 91%, 92%, 93%, 94%, 95%, 96%, 97%, 98%, or 99% identity to the antibody heavy chain of daratumumab, isatuximab, and felzartamab, trastuzumab, rituximab, brentuximab, cetuximab, panitumumab, GC33 (or a humanized version thereof), anti-EGFR antibody mAb806 (or a humanized version thereof), sibrotuzumab (BIBH1), or a fragment thereof. In some embodiments, the antigen-binding moiety has an antibody light chain with at least 90%, 91%, 92%, 93%, 94%, 95%, 96%, 97%, 98%, or 99% identity to the antibody light chain of daratumumab, isatuximab, and felzartamab, trastuzumab, rituximab, brentuximab, cetuximab, panitumumab, GC33 (or a humanized version thereof), anti-EGFR antibody mAb806 (or a humanized version thereof), sibrotuzumab (BIBH1), or a fragment thereof.

In some embodiments, the antigen-binding moiety comprises the six complementarity determining regions (CDRs) of daratumumab, isatuximab, and felzartamab, trastuzumab, rituximab, brentuximab, cetuximab, panitumumab, GC33, anti-EGFR antibody mAb806, or sibrotuzumab (BIBH1). A number of CDR delineations are known in the art and are encompassed herein. A person of skill in the art can readily determine a CDR for a given delineation based on the sequence of the heavy or light chain variable region. The “Kabat” Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). “Chothia” CDRs refer to the location of the structural loops (Chothia & Lesk, J. Mol. Biol. (1987) 196:901-917). The “AbM” CDRs represent a compromise between the Kabat CDRs and Chothia structural loops, and are used by Oxford Molecular's AbM antibody modeling software. The “Contact” CDRs are based on an analysis of the available complex crystal structures. The residues from each of these CDRs are noted below in Table 1, in reference to common antibody numbering schemes. Unless otherwise specified herein, amino acid number of antibodies refers to the Kabat numbering scheme as described in Kabat et al., supra, including when CDR delineations are made in reference to Kabat, Chothia, AbM, or Contact schemes. Using this numbering system, the actual linear amino acid sequence may contain fewer or additional amino acids corresponding to a shortening of, or insertion into, a framework region (FR) or CDR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

TABLE 1
CDR Delineations According to Various Schemes

| CDR | Kabat | AbM | Chothia | Contact |
|---|---|---|---|---|
| VL-CDR1 | L24-L34 | L24-L34 | L26-L32 | L30-L36 |
| VL-CDR2 | L50-L56 | L50-L56 | L50-L52 | L46-L55 |
| VL-CDR3 | L89-L97 | L89-L97 | L91-L96 | L89-L96 |
| VH-CDR1 | H31-H35B | H26-H35B | H26-H32 | H30-H35B |
| (Kabat | | | | |
| Numbering) | | | | |
| VH-CDR1 | H31-H35 | H26-H35 | H26-H32 | H30-H35 |
| (Chothia | | | | |
| Numbering) | | | | |
| VH-CDR2 | H50-H65 | H50-H58 | H53-H55 | H47-H58 |
| VH-CDR3 | H95-H102 | H95-H102 | H95-H101 | H93-H101 |

In some embodiments, the CDRs are “extended CDRs,” and encompass a region that begins or terminates according to a different scheme. For example, an extended CDR can be as follows: L24-L36, L26-L34, or L26-L36 (VL-CDR1); L46-L52, L46-L56, or L50-L55 (VL-CDR2); L91-L97 (VL-CDR3); H47-H55, H47-H65, H50-H55, H53-H58, or H53-H65 (VH-CDR2); and/or H93-H102 (VH-CDR3).

In some embodiments, the IFN prodrug of the present disclosure comprises a carrier comprising an antigen-binding moiety; wherein the antigen-binding moiety binds to Trop-2. In some embodiments, the IFN prodrug is used to treat patients with solid tumors. In some embodiments, the prodrug is used to treat triple negative breath cancer, urothelial cancer, small-cell lung cancer, pancreatic cancer, hilar cholangiocarcinoma, cervical cancer, and gastric cancer.

In some embodiments, the IFN prodrug comprises a carrier moiety comprising an antigen-binding moiety; wherein the antigen-binding moiety binds to 5T4. In some embodiments, the IFN prodrug is used to treat patients with solid tumor. In some embodiments, the prodrug is used to treat triple negative breath cancer, small-cell lung cancer, non-small cell lung cancer, pancreatic cancer, ovarian cancer, and gastric cancer.

In some embodiments, the IFN prodrug comprises a carrier moiety comprising an antigen-binding moiety, wherein the antigen-binding moiety binds to Claudin 18.2. In some embodiments, the IFN prodrug is used to treat patients with pancreatic cancer and gastric cancer.

In some embodiments, the IFN prodrug comprises a carrier comprising an antigen-binding moiety, wherein the antigen-binding moiety binds to EGFR Type III. In some embodiments, the IFN is used to treat patients with lung cancer, glioblastoma, and colon cancer.

In some embodiments, the IFN prodrug comprises a carrier comprising an antigen-binding moiety, wherein the antigen-binding moiety binds to CD38 or BCMA. In some embodiments, the IFN is used to treat patients with multiple myeloma.

In some embodiments, the IFN prodrugs are used in combination with an immune checkpoint blockade, such as a PD-1 antibody or an PD-1 antibody fragment thereof.

C. Linker Components of the Prodrugs

The IFN agonist polypeptide (cytokine moiety) may be fused to the carrier moiety with or without a peptide linker. The peptide linker may be non-cleavable and may be selected from the following sequences: GGGGS (SEQ ID NO: 122), GGGGSGGGGS (SEQ ID NO: 123), GGGGSGGGGSGGGGS (SEQ ID NO: 124), or GGGGSGGGGSAAGGGGSGGGGS (SEQ ID NO: 125).

The mask moiety may be fused to the cytokine moiety, to the carrier, or to another mask through a non-cleavable linker or a cleavable linker. The cleavable linker may contain one or more (e.g., two or three) cleavable moieties (CM). Each CM may be a substrate for an enzyme or protease selected from legumain, plasmin, TMPRSS-3/4, matrix metallopeptidase 2 (MMP2), MMP9, matrix metallopeptidase (MT1-MMP), cathepsin, caspase, human neutrophil elastase, beta-secretase, uPA, and PSA. Examples of cleavable linkers include, without limitation, those comprising an amino acid sequence selected from SEQ ID NOs: 26-45.

### II. Examples of IFN Prodrugs

In some embodiments, the IFN prodrug comprises an antibody or an Fc domain, and an IFN agonist polypeptide, wherein the IFN agonist polypeptide is fused to the C-terminal of one of the heavy chains of the antibody or one of the Fc polypeptide chains of the Fc domain, optionally through a peptide linker; wherein the masking moiety is fused to the C-terminal of the other heavy chain or other Fc polypeptide chain, through a cleavable peptide linker. In some embodiments, the cytokine components and the masking moieties are located on the N-termini of the Fc domains.

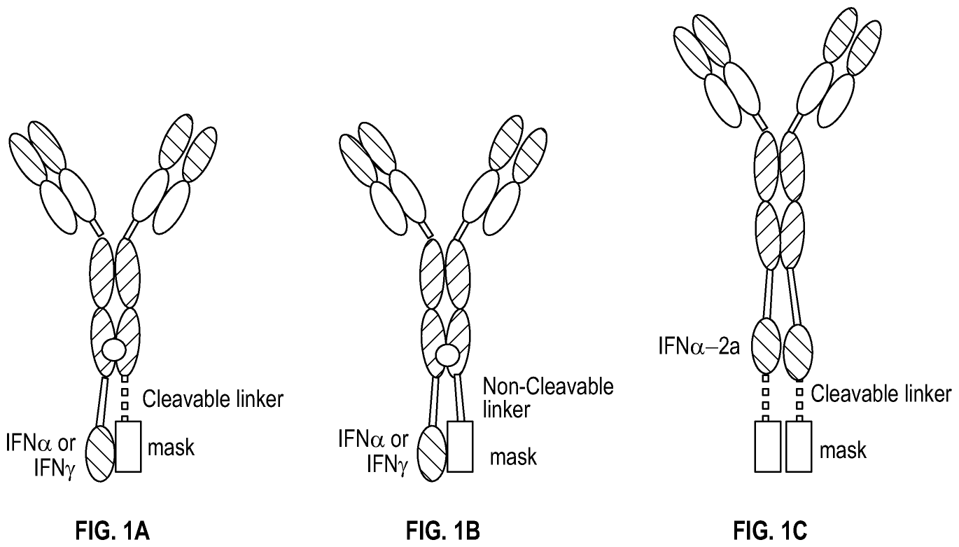

By way of example, an IFNα prodrug comprises a structure as shown in FIG. 1A, 1B or 1C. FIG. 1A shows an activatable IFNα prodrug with an antibody as its carrier and a cleavable linker, wherein the prodrug comprises two identical light chains, a heavy chain-mask fusion polypeptide chain comprising a cleavable linker, and a heavy chain-cytokine fusion polypeptide chain. FIG. 1B shows an IFNα prodrug with an antibody as its carrier, wherein the prodrug comprises two identical light chains, a heavy chain-mask fusion polypeptide chain comprising a non-cleavable linker, and a heavy chain-cytokine fusion polypeptide chain. FIG. 1C shows an activatable IFNα prodrug with an antibody as its carrier and cleavable linkers, wherein the prodrug comprises two identical light chains and two identical heavy chain polypeptide chains; wherein each of the heavy chain polypeptide chains comprises a cytokine fused to the C-terminus of the heavy chain and a mask fused to the C-terminus of each cytokine through a cleavable linker.

By way of example, the INFγ prodrug comprises a first polypeptide chain and a second polypeptide chain; wherein the first polypeptide chain comprises an amino acid sequence selected from SEQ ID NOs: 14, 15, 16, and 17 or at least 90%, at least 95%, or at least 99% identical to SEQ ID NO: 14, 15, 16, or 17, and the second polypeptide chain comprises an amino acid sequence selected from SEQ ID NOs: 18 and 19 or at least 90%, at least 95%, or at least 99% identical to SEQ ID NO: 18 or 19.

By way of example, the INFγ prodrug comprises a first polypeptide chain and a second polypeptide chain; wherein the first polypeptide chain comprises an amino acid sequence selected from SEQ ID NOs: 20, 21, 22, and 23 or at least 90%, at least 95%, or at least 99% identical to SEQ ID NO: 20, 21, 22, or 23, and the second polypeptide chain comprises an amino acid sequence selected from SEQ ID NOs: 24 and 25 or at least 90%, at least 95%, or at least 99% identical to SEQ ID NO: 24 or 25.

By way of example, the INFγ prodrug comprises two identical light chains, a first heavy chain polypeptide chain and a second heavy chain polypeptide chain; wherein the light chain comprises an amino acid sequence of SEQ ID NO:46 or at least 95% identical to SEQ ID NO: 46, the first heavy chain polypeptide chain comprises an amino acid sequence selected from SEQ ID NOs: 47, 48, 49, and 50 or at least 90%, at least 95%, or at least 99% identical to SEQ ID NOs: 47, 48, 49, or 50, and the second polypeptide chain comprises an amino acid sequence selected from SEQ ID NOs: 51 and 52 or at least 90%, at least 95%, or at least 99% identical to SEQ ID NO: 51, or 52.

By way of example, the INFγ prodrug comprises two identical light chains, a first heavy chain polypeptide chain and a second heavy chain polypeptide chain; wherein the light chain comprises an amino acid sequence of SEQ ID NO:53 or at least 95% identical to SEQ ID NO: 53, the first heavy chain polypeptide chain comprises an amino acid sequence selected from SEQ ID NOs: 54, 55, 56, and 57 or at least 90%, at least 95%, or at least 99% identical to SEQ ID NO: 54, 55, 56, or 57, and the second polypeptide chain comprises an amino acid sequence selected from SEQ ID NOs: 58 and 59 or at least 90%, at least 95%, or at least 99% identical to SEQ ID NO: 58, or 59.

By way of example, the INFα prodrug comprises a first polypeptide chain and a second polypeptide chain; wherein said first polypeptide chain comprises an amino acid sequence selected from SEQ ID NOs: 67, 68, 69, and 70 or at least 90%, at least 95%, or at least 99% identical to SEQ ID NO: 67, 68, 69, or 70, and the second polypeptide chain comprises an amino acid sequence selected from SEQ ID NOs: 71 and 72 or at least 90%, at least 95%, or at least 99% identical to SEQ ID NO: 71 or 72.

By way of example, the INFα prodrug comprises a first polypeptide chain and a second polypeptide chain; wherein the first polypeptide chain comprises an amino acid sequence selected from SEQ ID NOs: 73, 74, 75, and 76 or at least 90%, at least 95%, or at least 99% identical to SEQ ID NO: 73, 74, 75, or 76, and the second polypeptide chain comprises an amino acid sequence selected from SEQ ID NO: 77, 78 or at least 90%, at least 95%, or at least 99% identical to SEQ ID NO: 77 or 78.

By way of example, the INFα prodrug comprises two identical light chains, a first heavy chain polypeptide chain and a second heavy chain polypeptide chain; wherein the light chain comprises an amino acid sequence of SEQ ID NO: 46 or at least 90%, at least 95%, or at least 99% identical to SEQ ID NO: 46, the first heavy chain polypeptide chain comprises an amino acid sequence selected from SEQ ID NOs: 79, 80, 81, and 82 or at least 90%, at least 95%, or at least 99% identical to SEQ ID NO: 79, 80, 81, or 82, and the second polypeptide chain comprises an amino acid sequence selected from SEQ ID NOs:83 and 84 or at least 90%, at least 95%, or at least 99% identical to SEQ ID NO: 83 or 84.

By way of example, the INFα prodrug comprises two identical light chains, a first heavy chain polypeptide chain and a second heavy chain polypeptide chain; wherein the light chain comprises an amino acid sequence of SEQ ID NO: 53 or at least 95% identical to SEQ ID NO: 53, the first heavy chain polypeptide chain comprises an amino acid sequence selected from SEQ ID NOs:85, 86, 87, and 88 or at least 90%, at least 95%, or at least 99% identical to SEQ ID NO: 85, 86, 87, or 88, and the second polypeptide chain comprises an amino acid sequence selected from SEQ ID NOs: 89 and 90 or at least 90%, at least 95%, or at least 99% identical to SEQ ID NO: 89 or 90.

By way of example, the INFα prodrug comprises two identical light chains, a first heavy chain polypeptide chain and a second heavy chain polypeptide chain; wherein the light chain comprises an amino acid sequence of SEQ ID NO: 120 or at least 95% identical to SEQ ID NO: 120, the first heavy chain polypeptide chain comprises an amino acid sequence selected from SEQ ID NOs: 113 and 114 or at least 98% identical to SEQ ID NO: 113 or 114, and the second polypeptide chain comprises an amino acid sequence selected from SEQ ID NOs: 115, 116, 117, 118, and 119 or at least 98% identical to SEQ ID NO: 115, 116, 117, 118, or 119.

By way of example, the INFα prodrug comprises two identical light chains and two identical heavy chain polypeptide chains; wherein the light chain comprises an amino acid sequence of SEQ ID NO: 94 or at least 95% identical to SEQ ID NO: 94, and the heavy chain polypeptide chain comprises an amino acid sequence SEQ ID NO: 126. By way of example, the INFα the prodrug comprises two identical light chains, a two identical heavy chain polypeptide chains; wherein said light chain comprises an amino acid sequence of SEQ ID NO: 96 or at least 95% identical to 96, said heavy chain polypeptide chain comprises an amino acid sequence of SEQ ID NO:127 or at least 98% identical to SEQ ID NO:127.

### III. Pharmaceutical Compositions

Pharmaceutical compositions of the prodrugs are prepared by mixing the presently disclosed prodrugs, or antibody fusion molecules or the antibody fusion molecule drug conjugate having the desired degree of purity with one or more optional pharmaceutically acceptable carriers (see Osol, A. Ed. Remington's Pharmaceutical Sciences 16th edition (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The choice of pharmaceutical carrier, excipient or diluent may be selected with regard to the intended route of administration and standard pharmaceutical practice. Pharmaceutical compositions may comprise as—or in addition to—the carrier, excipient, or diluent any suitable binder(s), lubricant(s), suspending agent(s), coating agent(s) or solubilizing agent(s).

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular, or subcutaneous route. Alternatively, the formulation may be designed to be administered by a number of routes. In some embodiments, said formulation is administrated directly in a tumor or tumors.

In some embodiments, an antibody or protein formulation is a lyophilized formulation. In another embodiments, an antibody or protein formulation is an aqueous formulation.

In some embodiments, the pharmaceutical composition is a combination pharmaceutical composition, which comprises an IFN prodrug of the present invention, a pharmaceutically acceptable excipient, and a second active ingredient selected from a cytokine other than IFN or its prodrug or fusion molecule, an antibody against PD-1, an antibody against PD-L1, an antibody against CTLA-4, an antibody against CD47, a PD-1 antibody-IL-15 fusion molecule, a PD-1-IL-2 fusion molecule, and a PD-1-IL-21 fusion molecule.

### IV. Methods of Treatment

The presently disclosed prodrugs can be used to treat a disease, depending on the antigen bound by the antigen-binding moiety. In some embodiments, the prodrugs disclosed herein are used to treat cancer. In some embodiments, the prodrugs are used to treat an infection, for example when the drug molecule is an antibacterial agent or an antiviral agent.

In some embodiments, a method of treating a disease (such as cancer, a viral infection, or a bacterial infection) in a subject comprises administering to the subject an effective amount of the presently disclosed prodrugs.

In some embodiments, the cancer is a solid cancer. In some embodiments, the cancer is a blood cancer. Exemplary cancers that may be treated include, but are not limited to, leukemia, lymphoma, kidney cancer, bladder cancer, urinary tract cancer, cervical cancer, brain cancer, head and neck cancer, skin cancer, uterine cancer, testicular cancer, esophageal cancer, liver cancer, colorectal cancer, stomach cancer, squamous cell carcinoma, prostate cancer, pancreatic cancer, lung cancer, cholangiocarcinoma, breast cancer, and ovarian cancer.

In some embodiments, the presently disclosed prodrugs are used to treat a bacterial infection such as sepsis. In some embodiments, the bacteria causing the bacterial infection are drug-resistant bacteria. In some embodiments, the antigen-binding moiety binds to a bacterial antigen.

In some embodiments, the prodrug is used to treat a viral infection. In some embodiments, the virus causing the viral infection is hepatitis C (HCV), hepatitis B (HBV), human immunodeficiency virus (HIV), a human papilloma virus (HPV). In some embodiments, the antigen-binding moiety binds to a viral antigen.

Generally, dosages and routes of administration of the present pharmaceutical compositions are determined according to the size and condition of the subject, according to standard pharmaceutical practice. In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally. In some embodiments, the composition is administered to a subject intravenously.

In some embodiments, the prodrug is administered to a subject in need a single dose or a repeated dose. In some embodiments, the doses are given to a subject once per day, twice per day, three times per day, or four or more times per day. In some embodiments, about 1 or more (such as about 2, 3, 4, 5, 6, or 7 or more) doses are given in a week. In some embodiments, the antibody fusion molecule conjugated to the drug is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2, 2, 3, 4, 5, 7, 10, 15, or 20 or more doses).

In some embodiments, the IFN prodrug is administered to a subject in combination with a second pharmaceutical composition, wherein the second pharmaceutical composition comprises an active ingredient selected from a cytokine other than IFN or its prodrug or fusion molecule, an antibody against PD-1, an antibody against PD-L1, an antibody against CTLA-4, an antibody against CD47, a PD-1 antibody-IL-2 fusion molecule, a PD-1-IL-7 fusion molecule, a PD-1 antibody-IL-15 fusion molecule, and a PD-1-IL-21 fusion molecule.

### V. Methods of Making the Prodrugs

The presently disclosed prodrugs can be produced using recombinant DNA methods. Nucleic acid molecules encoding the polypeptide or the fusion polypeptide of said prodrug can be isolated and inserted into one or more vectors for further cloning and/or expression in a host cell. Such nucleic acid molecules may be readily isolated and sequenced using conventional methods. Suitable host cells for cloning or expression of fusion polypeptide vectors include prokaryotic cells or eukaryotic cells. Exemplary host cells include Chinese Hamster Ovary (CHO) cells or human embryonic kidney cells (e.g., HEK293).

Expression host cells express the antibody fusion molecule. After an expression period, the host cells can by lysed and the prodrug or antibody fusion molecule can be purified. Exemplary purification methods include liquid chromatography, such as ion exchange chromatography, affinity chromatography (such as Protein A affinity chromatography), or size exclusion chromatography.

### VI. Exemplary Embodiments

Further particular embodiments of the present disclosure are described as follows. These embodiments are intended to illustrate the compositions and methods described in the present disclosure and are not intended to limit the scope of the present disclosure.

1. A prodrug of Interferon gamma (IFNγ), which comprises an IFNγ agonist polypeptide and a masking moiety; wherein said masking moiety binds to IFNγ and inhibits a biological activity of IFNγ; and wherein said IFNγ comprises an amino acid sequence selected from SEQ ID NO: 5, 6, and 7, or at least 90% identical as that of SEQ ID NO: 5, 6, or 7.

2. Prodrug of embodiment 1, wherein said masking moiety is selected from Interferon gamma Receptor 1 extracellular domain (IFGR1-ECD) or a functional analog thereof, or an antibody or a binding fragment thereof which binds to IFNγ.

3. Prodrug of embodiment 1, 2, or 3, wherein said masking moiety comprises IFNGR1-ECD or a functional analog thereof; and wherein said IFNGR1-ECD comprises an amino acid sequence selected from SEQ ID NOs: 8 and 9.

4. Prodrug of embodiment 1, 2, or 3, wherein said masking moiety comprises a scFv which comprises an amino acid sequence selected from SEQ ID NOs: 1-4, or at least 90% identical as SEQ ID NOs: 1, 2, 3, or 4.

5. Prodrug of embodiment 1, 2, 3, or 4, which further comprises a carrier moiety, which is selected from an Fc domain, an antigen-binding moiety, or an albumin or a fragment thereof.

6. Prodrug of embodiment 1, 2, 3, or 4, which further comprises a carrier moiety, wherein said carrier moiety comprises an Fc domain of an antibody.

7. Prodrug of embodiment 1, 2, 3, or 4, which further comprises a carrier moiety, wherein said carrier moiety comprises an antibody which bind to an antigen expressed on the surface of a tumor cell, a cancer cell, or an immune cell.

8. Prodrug of embodiment 7, wherein said immune cell is selected from an NK cell, a T cell, a B cell, and a macrophage.

9. Prodrug of embodiment 1, 2, 3, or 4, which further comprises a carrier moiety, wherein said carrier moiety comprises an antibody which bind to an antigen selected from PD-1, LAG-3, SIRPα, ILT2, CD206, NKD2G, CTLA-4, CD8, and CD16a.

10. Prodrug of embodiment 1, 2, 3, or 4, which further comprises a carrier moiety, wherein said carrier moiety comprises an antibody which bind to an antigen selected from PD-L1, CD47, CMET, EGFR, ROR1, TROP-2, HER2, CLDN18.2, and VEGFR2.

11. Prodrug of embodiment 1, 2, 3, or 4, which further comprises a carrier moiety, wherein said carrier moiety comprises an antibody which bind to PD-1; and wherein said antibody comprises a light chain variable domain with an amino acid sequence of SEQ ID NO: 10 and a heavy chain variable domain with an amino acid sequence of SEQ ID NO: 11, or a light chain variable domain with an amino acid sequence of SEQ ID NO: 12 and a heavy chain variable domain with an amino acid sequence of SEQ ID NO: 13.

12. Prodrug of any of embodiments 1-11, which further comprises a cleavable peptide linker, which is cleavable by enzyme expressed in or near a tumor.

13. Prodrug of embodiment 6, wherein said cleavable peptide linker comprises an amino acid sequence selected from SEQ ID NOs: 26-45.

14. A prodrug of INFγ, which comprises a first polypeptide chain and a second polypeptide chain; wherein said first polypeptide chain comprises an amino acid sequence selected from SEQ ID NOs: 14, 15, 16, and 17 or at least 95% identical as that of SEQ ID NO: 14, 15, 16, or 17, and said second polypeptide chain comprises an amino acid sequence selected from SEQ ID NOs: 18 and 19 or at least 95% identical as that of SEQ ID NO: 18 or 19.

15. A prodrug of INFγ, which comprises a first polypeptide chain and a second polypeptide chain; wherein said first polypeptide chain comprises an amino acid sequence selected from SEQ ID NOs: 20, 21, 22, and 23 or at least 95% identical as that of SEQ ID NO: 20, 21, 22, or 23, and said second polypeptide chain comprises an amino acid sequence selected from SEQ ID NOs: 24 and 25 or at least 95% identical as that of SEQ ID NO: 24 or 25.

16. A prodrug of INFγ, which comprises two identical light chains, a first heavy chain polypeptide chain and a second heavy chain polypeptide chain; wherein said light chain comprises an amino acid sequence of SEQ ID NO: 46 or at least 95% identical as that of 46, said first heavy chain polypeptide chain comprises an amino acid sequence selected from SEQ ID NOs: 47, 48, 49, and 50 or at least 98% identical as that of SEQ ID NOs: 47, 48, 49, or 50, and said second polypeptide chain comprises an amino acid sequence selected from SEQ ID NOs: 51 and 52 or at least 98% identical as that of SEQ ID NO: 51, or 52.

17. A prodrug of INFγ, which comprises two identical light chains, a first heavy chain polypeptide chain and a second heavy chain polypeptide chain; wherein said light chain comprises an amino acid sequence of SEQ ID NO: 53 or at least 95% identical as that of 53, said first heavy chain polypeptide chain comprises an amino acid sequence selected from SEQ ID NOs: 54, 55, 56, and 57 or at least 98% identical as that of SEQ ID NO: 54, 55, 56, or 57, and said second polypeptide chain comprises an amino acid sequence selected from SEQ ID NOs: 58 and 59 or at least 98% identical as that of SEQ ID NO: 58, or 59.

18. A prodrug of interferon alpha (INFα), which comprises an INFα agonist polypeptide, a masking moiety, and a carrier moiety; wherein said masking moiety comprises a Fab, a nanobody, or a single chain Fv (scFv) which binds to said INFα agonist.

19. Prodrug of embodiment 18, wherein said INFα comprises an amino acid sequence selected from SEQ ID NOs: 65 and 66 or at least 95% identical as that of SEQ ID NO: 65 or 66.

20. Prodrug of embodiment 18 or 19, wherein said masking moiety comprises a scFv, which comprises the same heavy chain CDR1, CDR2, CDR3, and light chain CDR1, CDR2, CDR3 as derived from antibody sifalimumab or rontalizumab.

21. Prodrug of embodiment 18 or 19, wherein said masking moiety comprises a scFv, which comprises a VL domain with an amino acid sequence of SEQ ID NO: 63 or at least 95% identical as that of SEQ ID NO: 63, and a VH domain with an amino acid sequence of SEQ ID NO: 64 or at least 95% identical as that of SEQ ID NO: 64.

22. Prodrug of embodiment 18 or 19, wherein said masking moiety comprises a scFv, which comprises an amino acid sequence of SEQ ID NO: 60 or 61 or at least 95% identical as that of SEQ ID NO: 60 or 61.

23. Prodrug of any of embodiments 18-22, wherein said carrier moiety is selected from an Fc domain, an antigen-binding moiety, or an albumin or a fragment thereof.

24. Prodrug of any of embodiments 18-22, wherein said carrier moiety comprises an antibody which bind to an antigen expressed on the surface of a tumor cell, a cancer cell, or an immune cell.

25. Prodrug of embodiment 24, wherein said immune cell is selected from an NK cell, a T cell, a B cell, and a macrophage.

26. Prodrug of any of embodiments 18-22, wherein said carrier moiety comprises an antibody which bind to an antigen selected from PD-1, LAG-3, SIRPα, ILT2, CD206, NKD2G, CTLA-4, CD8, and CD16a.

27. Prodrug of any of embodiments 18-22, wherein said carrier moiety comprises an antibody which bind to an antigen selected from PD-L1, CD47, CMET, EGFR, ROR1, TROP-2, HER2, CLDN18.2, and VEGFR2.

28. Prodrug of any of embodiments 18-22, wherein said carrier moiety comprises an antibody which bind to PD-1; and wherein said antibody comprises a light chain variable domain with an amino acid sequence of SEQ ID NO: 10 and a heavy chain variable domain with an amino acid sequence of SEQ ID NO: 11, or a light chain variable domain with an amino acid sequence of SEQ ID NO: 12 and a heavy chain variable domain with an amino acid sequence of SEQ ID NO: 13.

29. Prodrug of any of embodiments 18-28, which further comprises a cleavable peptide linker, which is cleavable by enzyme expressed in or near a tumor.

30. Prodrug of embodiment 29, wherein said cleavable peptide linker comprises an amino acid sequence selected from SEQ ID NOs: 26-45.

31. A prodrug of INFα, which comprises a first polypeptide chain and a second polypeptide chain; wherein said first polypeptide chain comprises an amino acid sequence selected from SEQ ID NOs: 67, 68, 69, and 70 or at least 95% identical as that of SEQ ID NO: 67, 68, 69, or 70, and said second polypeptide chain comprises an amino acid sequence selected from SEQ ID NOs: 71 and 72 or at least 95% identical as that of SEQ ID NO: 71 or 72.

32. A prodrug of INFα, which comprises a first polypeptide chain and a second polypeptide chain; wherein said first polypeptide chain comprises an amino acid sequence selected from SEQ ID NOs: 73, 74, 75, and 76 or at least 95% identical as that of SEQ ID NO: 73, 74, 75, or 76, and said second polypeptide chain comprises an amino acid sequence selected from SEQ ID NO: 77, 78 or at least 95% identical as that of SEQ ID NO: 77 or 78.

33. A prodrug of INFα, which comprises two identical light chains, a first heavy chain polypeptide chain and a second heavy chain polypeptide chain; wherein said light chain comprises an amino acid sequence of SEQ ID NO: 46 or at least 95% identical as that of 46, said first heavy chain polypeptide chain comprises an amino acid sequence selected from SEQ ID NOs: 79, 80, 81, and 82 or at least 98% identical as that of SEQ ID NO: 79, 80, 81, or 82, and said second polypeptide chain comprises an amino acid sequence selected from SEQ ID NOs: 83 and 84 or at least 98% identical as that of SEQ ID NO: 83 or 84.

34. A prodrug of INFα, which comprises two identical light chains, a first heavy chain polypeptide chain and a second heavy chain polypeptide chain; wherein said light chain comprises an amino acid sequence of SEQ ID NO: 53 or at least 95% identical as that of 53, said first heavy chain polypeptide chain comprises an amino acid sequence selected from SEQ ID NOs: 85, 86, 87, and 88 or at least 98% identical as that of SEQ ID NO: 85, 86, 87, or 88, and said second polypeptide chain comprises an amino acid sequence selected from SEQ ID NOs: 89 and 90 or at least 98% identical as that of SEQ ID NO: 89 or 90.

35. A pharmaceutical composition comprising the prodrug of any one of embodiments 1-34 as its active ingredient, and a pharmaceutically acceptable excipient.

36. The pharmaceutical composition of embodiment 35, which further comprises an antagonist of CD47, an antagonist of SIRPα, or an antagonist of CTLA4.

37. A polynucleotide or polynucleotides encoding the chimeric molecule of any one of embodiments 1-34.

38. An expression vector or vectors comprising the polynucleotide or polynucleotides of embodiment 37.

39. A host cell comprising the vector(s) of embodiment 38.

40. The host cell of embodiment 39, wherein the host cell has the gene or genes encoding uPA, MMP-2, MMP-9 and/or matriptase are knocked out.

41. A method of making the chimeric molecule of any one of embodiments 1-34, comprising culturing the host cell of embodiment 39 or 40 under conditions that allow expression of the chimeric molecule, and isolating the chimeric molecule.

42. A method of treating a cancer or an infectious disease or stimulating the immune system in a patient in need thereof, comprising administering the prodrug of any one of claims 1-34 or the pharmaceutical composition of embodiment 35 or 36.

43. A method of treating a cancer in a patient in need thereof, comprising administering the the prodrug of any one of claims 1-34 or pharmaceutical composition of embodiment 35, wherein said patient is also administrated with a pharmaceutical composition which comprises an antagonist of CD47, an antagonist of ILT2, an antagonist of SIRPα, an antibody against PD-1, an antibody against CTLA-4, or an antibody against PD-1.

44. A method of treating of a patient with cancer, comprising administering the prodrug of any one of claims 1-34 or pharmaceutical composition of embodiment 35 or 36 directly into a tumor or tumors.

45. The method of embodiment 42, 43, or 44, wherein said cancer is selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer.

It is understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is not intended to be limited to a particular compound, composition, article, or method, unless expressly stated as such. In addition, those of ordinary skill in the art will recognize that certain changes, modification, permutations, alterations, additions, subtractions, and sub-combinations thereof can be made in accordance with the teachings herein without departing from the spirit of the present specification.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: Transient Transfection of ExpiCHO Cells

Expression plasmids were co-transfected into 6×10⁶ cell/ml freestyle ExpiCHO cells at 1 μg/ml using ExpiFectamine™ CHO Transfection kit (Gibco). For an antibody, the HC and LC ratio was 1:2. For an antibody-fused IFNα, prodrug, the HC-IFNα fusion polypeptide, the HC-masking moiety fusion polypeptide, and the LC were in a 1:1.5:4 ratio. Table 2 shows the sequence IDs of the 5T4 antibody JR11.60.1 and 5T4 antibody-IFNα prodrug molecules JR11.60.2 and JR11.60.3 expressed. The cell cultures were harvested 7 days after transfection by centrifuging at 9,000 rpm for 45 min followed by 0.22 μM filtration.

TABLE 2
SEQ ID NO's used in the transient transfection
expression of the IFNα prodrug.

| | Chain 1 - | Chain 2 - | |
|---|---|---|---|
| Molecule | HC or HC- | HC- Mask | Chain 3 - |
| Name | IFNα Fusion | Fusion | Light Chain |
| JR11.60.1 | SEQ ID NO: 121 | — | SEQ ID NO: 120 |
| JR11.60.2 | SEQ ID NO: 118 | SEQ ID NO: 113 | SEQ ID NO: 120 |
| JR11.60.3 | SEQ ID NO: 119 | SEQ ID NO: 113 | SEQ ID NO: 120 |

### Example 2: Purification of IFNα Prodrugs

The purifications of the proteins of the IFNα prodrugs were carried out using Protein A affinity chromatography. Additional purification steps are carried out using additional chromatography and filtration steps. For example, chromatography steps with resins such as Capto™ MMC ImpRes, Capto™ Adhere, Capto™ SP, and/or Q Sepharose FF can be used for further purification of the prodrugs.

### Example 3: SEC-HPLC Analysis

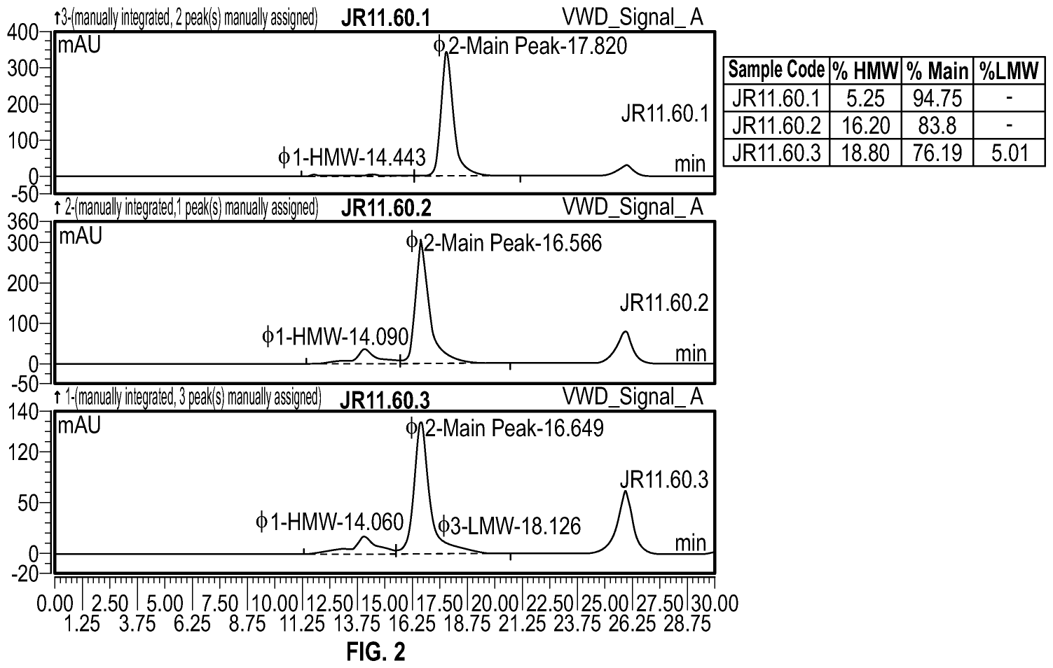

SEC-HPLC was carried out using an Agilent 1100 Series of HPLC system with a TSKgel G3000SWXL column (7.8 mmID×30 cm, 5 μm particle size) ordered from Tosoh Bioscience. A sample of up to 100 μl was loaded. The column was run with a buffer containing 200 mM K₃PO₄, 250 mM KCl, pH 6.5. The flow rate was 0.5 ml/min. The column was run at room temperature. The protein elution was monitored both at 220 nm and 280 nm. FIG. 2 shows the SEC-HPLC data of the 5T4 antibody JR11.60.1 and prodrugs JR11.60.2 and JR11.60.3 after Protein A affinity chromatography purification.

### Example 4: SDS-PAGE Analysis

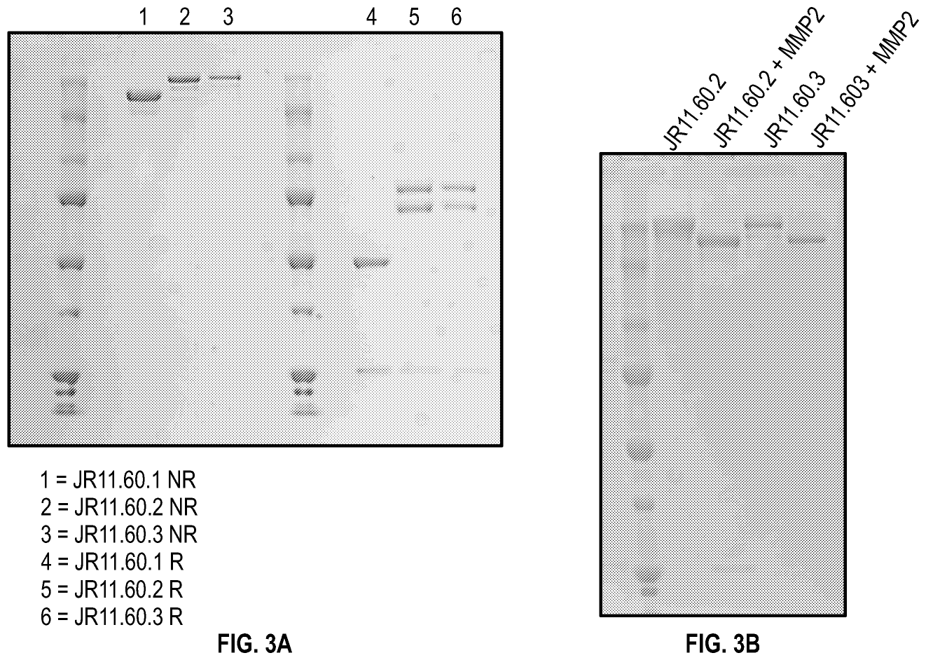

10 μl of the culture supernatants or 4 μg of purified protein samples were mixed with Bolt™ LDS Sample Buffer (Novex) with or without reduce reagents. The samples were heated at 70° C. for 3 min and then loaded to a NuPAGE™ 4-12% BisTris Gel (Invitrogen™). The gel was run in NuPAGE™ MOPS SDS Running buffer (Invitrogen™) at 200 Volts for 35 min and then stained with Coomassie. FIG. 3A shows the non-reduced and reduced SDS-PAGE analysis of JR11.60.1, JR11.60.2 and JR11.60.3 after Protein A affinity chromatography purification. The light chain and heavy chain polypeptide chains showed expected molecule weights in the reduced SDS-PAGE.

### Example 5: Proteolytic Treatment

One μg of the protease, human MMP-2 (R&D systems), human MMP-9 (R&D systems), mouse MMP-2 (R&D systems), or mouse MMP-9 (R&D systems) was added to 50 μg of the precursor protein, and incubated at 37° C. overnight. The SDS-PAGE analysis of the activatable fusion molecules prior to and after activation is shown in FIG. 3B (non-reduced). Reductions of molecule weights were observed after the enzymatic digestion.

### Example 6: Anti-Proliferation Assay

The ability of Type I Interferons to inhibit proliferation of some tumor cell lines has long been known. There are many possible mechanisms for this activity including down regulation of the transcription factor c-myc and inhibiting the phosphorylation of the tumor suppressor protein pRb, both leading to cell cycle arrest. Treatment with IFNα can also result in up regulation of pro-apoptotic proteins such as Fas, FasL and TRAIL. To test the antiproliferative effects serial dilutions of test articles were performed in 96 well plates in 100 mL/well assay medium (RPMI 1640, 10% FBS, NEAA, Pyruvate, NEAA, beta-mercaptoethanol). Cell lines to be tested were added at 5,000 or 10,000 cells/well in 100 mL. Cultures were incubated for 3 days at 37° C., and 100 mL of culture supernatant was removed and replaced with 100 mL of CellTiter Glo® (Promega, Catalog #G9241). Luminescence was measured using a luminometer. CellTiter Glo® measures ATP activity provided quantitative detection of viable cells. Data were fitted with a four-parameter logistic (4PL) regression to give the EC₅₀ of the sample.

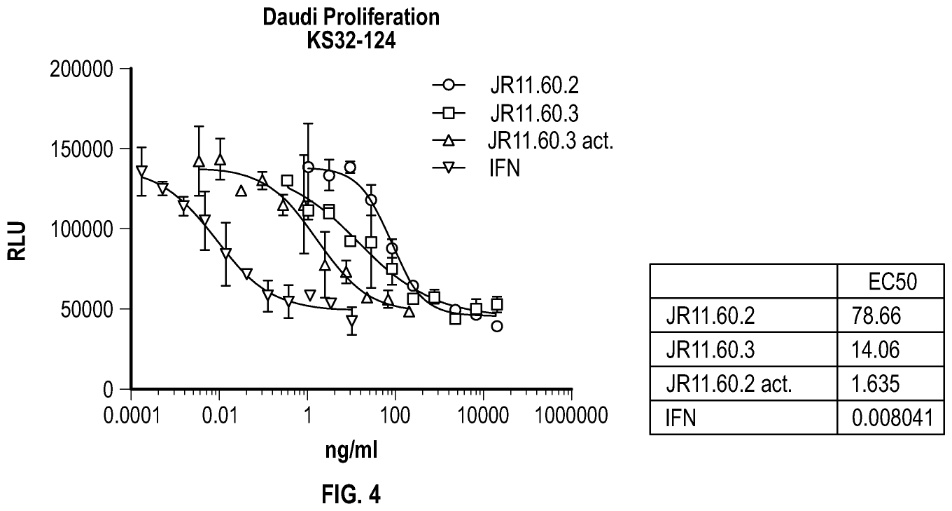

FIG. 4 shows the proliferation of Daudi cells at various concentrations of 5T4 antibody-IFNα prodrug molecules JR11.60.2 and JR11.60.3 as well as JR11.60.2 after activation. IFNα-2b was included as the positive control. The data showed that the biological activity of prodrug molecule JR11.60.2 increased by approximately 50 times after activation by the protease MMP-2. Surprisingly, both JR11.60.2 and the activated JR11.60.2 had significantly lower potency than the control IFNα-2b, indicating that the prodrug will be significantly safer than the wild type interferon.

The above non-limiting examples are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating cancer, a neurodegenerative, or an infectious disease.

SEQUENCES

SEQ ID NO: 1. scFv of HuZaf VL-VH
DIQMTQSPST LSASVGDRVT ITCKASENVD TYVSWYQQKP GKAPKLLIYG ASNRYTGVPS
RFSGSGSGTD FTLTISSLQP DDFATYYCGQ SYNYPFTFGQ GTKVEVKRGG GGSGGGGSGG
GGSQVQLVQS GAELKKPGSS VKVSCKASGY IFTSSWINWV KQAPGQGLEW IGRIDPSDGE
VHYNQDFKDK ATLTVDKSTN TAYMELSSLR SEDTAVYYCA RGELPWFADW GQGTLVTVSS
SEQ ID NO: 2. scFv of HuZaf VH-VL
QVQLVQSGAE LKKPGSSVKV SCKASGYIFT SSWINWVKQA PGQGLEWIGR IDPSDGEVHY
NQDEKDKATL TVDKSTNTAY MELSSLRSED TAVYYCARGF LPWFADWGRG TLVTVSSGGG
GSGGGGSGGG GSDIQMTQSP STLSASVGDR VTITCKASEN VDTYVSWYQQ KPGKAPKLLI
YGASNRYTGV PSRESGSGSG TDFTLTISSL QPDDFATYYC GQSYNYPFTE GQGTKVEVKR
SEQ ID NO: 3. scFv of AMG811 VL-VH
EIVLTQSPGT LSLSPGERAT LSCRASQSVS SSYLAWYQQK PGQAPRLLIY GASSRATGIP
DRESGSGSGT DETLTISRLE PEDFAVYYCQ RSGGSSFTFG PGTKVDIKGG GGSGGGGSGG
GGSEVQLVQS GAEVKKPGES LKISCKGSGY NFTSYWIGWV RQMPGKGLEL MGIIYPGDSD
TRYSPSFQGQ VTISADKSIS TAYLQWSSLK ASDTAMYYCG SGSYFYFDLW GRGTLVTVSS
SEQ ID NO: 4. scFv of AMG811 VH-VL
EVQLVQSGAE VKKPGESLKI SCKGSGYNFT SYWIGWVRQM PGKGLELMGI IYPGDSDTRY
SPSFQGQVTI SADKSISTAY LQWSSLKASD TAMYYCGSGS YFYFDLWGRG TLVTVSSGGG
GSGGGGSGGG GSEIVLTQSP GTLSLSPGER ATLSCRASQS VSSSYLAWYQ QKPGQAPRLL
IYGASSRATG IPDRESGSGS GTDETLTISR LEPEDFAVYY CQRSGGSSFT FGPGTKVDIK
SEQ ID NO: 5. Interferon gamma
QDPYVKEAEN LKKYFNAGHS DVADNGTLFL GILKNWKEES DRKIMQSQIV SFYFKLEKNE
KDDQSIQKSV ETIKEDMNVK FENSNKKKRD DFEKLTNYSV TDLNVQRKAI HELIQVMAEL
SPAAKTGKRK RSQMLERG
SEQ ID NO: 6. Interferon gamma 1b
CYCQDPYVKE AENLKKYFNA GHSDVADNGT LFLGILKNWK EESDRKIMQS QIVSFYFKLE
KNFKDDQSIQ KSVETIKEDM NVKFENSNKK KRDDFEKLTN YSVTDLNVQR KAIHELIQVM
AELSPAAKTG KRKRSQMLER GRRASQ
SEQ ID NO: 7. Interferon gamma with signal peptide
MKYTSYILAF QLCIVLGSLG CYCQDPYVKE AENLKKYFNA GHSDVADNGT LFLGILKNWK
EESDRKIMQS QIVSFYFKLF KNFKDDQSIQ KSVETIKEDM NVKFENSNKK KRDDFEKLTN
YSVTDLNVQR KAIHELIQVM AELSPAAKTG KRKRSQMLER GRRASQ
SEQ ID NO: 8. Interferon gamma Receptor 1 extracellular domain 1-108
(IFNGR1-ECD fragment)
EMGTADLGPS SVPTPTNVTI ESYNMNPIVY WEYQIMPQVP VETVEVKNYG VKNSEWIDAC
INISHHYCNI SDHVGDPSNS LWVRVKARVG QKESAYAKSE EFAVCRDGKI GPPMGTADLG
PSSVPTPTNV TIESYNMNPI VYWEYQIMPQ VPVFTVEVKN YGVKNSEWID ACINISHHYC
NISDHVGDPS NSLWVRVKAR VGQKESAYAK SEEFAVCRDG
SEQ ID NO: 9. Interferon gamma Receptor 1 extracellular domain 1-229
(IFNGR1-ECD)
EMGTADLGPS SVPTPTNVTI ESYNMNPIVY WEYQIMPQVP VETVEVKNYG VKNSEWIDAC
INISHHYCNI SDHVGDPSNS LWVRVKARVG QKESAYAKSE EFAVCRDGKI GPPKLDIRKE
EKQIMIDIFH PSVFVNGDEQ EVDYDPETTC YIRVYNVYVR MNGSEIQYKI LTQKEDDCDE
IQCQLAIPVS SLNSQYCVSA EGVLHVWGVT TEKSKEVCIT IFNSSIKGS
SEQ ID NO: 10. VL of nivolumab
EIVLTQSPAT LSLSPGERAT LSCRASQSVS SYLAWYQQKP GQAPRLLIYD ASNRATGIPA
RFSGSGSGTD FTLTISSLEP EDFAVYYCQQ SSNWPRTFGQ GTKVEIK
SEQ ID NO: 11. VH of nivolumab
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSS
SEQ ID NO: 12. VL of pembrolizumab
EIVLTQSPAT LSLSPGERAT LSCRASKGVS TSGYSYLHWY QQKPGQAPRL LIYLASYLES
GVPARFSGSG SGTDETLTIS SLEPEDFAVY YCQHSRDLPL TFGGGTKVEI K
SEQ ID NO: 13. VH of pembrolizumab
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YRFDMGEDYW GQGTTVTVSS
SEQ ID NO: 14. Fc-scFv HuZaf
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVCT LPPSREEMTK NQVSLSCAVK GFYPSDIAVE WESNGQPENN YKTTPPVLDS
DGSFFLVSKL TVDKSRWQQG NVESCSVMHE ALHNHYTQKS LSLSPGAGGG GSGGGGSGPL
GVRGGGGSGG GGSDIQMTQS PSTLSASVGD RVTITCKASE NVDTYVSWYQ QKPGKAPKLL
IYGASNRYTG VPSRESGSGS GTDFTLTISS LQPDDFATYY CGQSYNYPFT FGQGTKVEVK
RGGGGSGGGG SGGGGSQVQL VQSGAELKKP GSSVKVSCKA SGYIFTSSWI NWVKQAPGQG
LEWIGRIDPS DGEVHYNQDF KDKATLTVDK STNTAYMELS SLRSEDTAVY YCARGELPWE
ADWGQGTLVT VSS
SEQ ID NO: 15. Fc-scFv HuZaf
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVCT LPPSREEMTK NQVSLSCAVK GFYPSDIAVE WESNGQPENN YKTTPPVLDS
DGSFFLVSKL TVDKSRWQQG NVESCSVMHE ALHNHYTQKS LSLSPGAGGG GSGGGGSGPL
GVRGGGGSGG GGSQVQLVQS GAELKKPGSS VKVSCKASGY IFTSSWINWV KQAPGQGLEW
IGRIDPSDGE VHYNQDEKDK ATLTVDKSTN TAYMELSSLR SEDTAVYYCA RGFLPWFADW
GRGTLVTVSS GGGGSGGGGS GGGGSDIQMT QSPSTLSASV GDRVTITCKA SENVDTYVSW
YQQKPGKAPK LLIYGASNRY TGVPSRESGS GSGTDFTLTI SSLQPDDFAT YYCGQSYNYP
FTFGQGTKVE VKR
SEQ ID NO: 16. Fc-scFv AMG811
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVCT LPPSREEMTK NQVSLSCAVK GFYPSDIAVE WESNGQPENN YKTTPPVLDS
DGSFFLVSKL TVDKSRWQQG NVESCSVMHE ALHNHYTQKS LSLSPGAGGG GSGGGGSGPL
GVRGGGGSGG GGSEIVLTQS PGTLSLSPGE RATLSCRASQ SVSSSYLAWY QQKPGQAPRL
LIYGASSRAT GIPDRESGSG SGTDFTLTIS RLEPEDFAVY YCQRSGGSSF TFGPGTKVDI
KGGGGSGGGG SGGGGSEVQL VQSGAEVKKP GESLKISCKG SGYNFTSYWI GWVRQMPGKG
LELMGIIYPG DSDTRYSPSF QGQVTISADK SISTAYLQWS SLKASDTAMY YCGSGSYFYF
DLWGRGTLVT VSS
SEQ ID NO: 17. Fc-scFv AMG811
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVCT LPPSREEMTK NQVSLSCAVK GFYPSDIAVE WESNGQPENN YKTTPPVLDS
DGSFFLVSKL TVDKSRWQQG NVESCSVMHE ALHNHYTQKS LSLSPGAGGG GSGGGGSGPL
GVRGGGGSGG GGSEVQLVQS GAEVKKPGES LKISCKGSGY NFTSYWIGWV RQMPGKGLEL
MGIIYPGDSD TRYSPSFQGQ VTISADKSIS TAYLQWSSLK ASDTAMYYCG SGSYFYFDLW
GRGTLVTVSS GGGGSGGGGS GGGGSEIVLT QSPGTLSLSP GERATLSCRA SQSVSSSYLA
WYQQKPGQAP RLLIYGASSR ATGIPDRESG SGSGTDFTLT ISRLEPEDFA VYYCQRSGGS
SFTFGPGTKV DIK
SEQ ID NO: 18. IFNγ-Fc
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVYT LPPCREEMTK NQVSLWCLVK GFYPSDIAVE WESNGQPENN YKTTPPVLDS
DGSFFLYSKL TVDKSRWQQG NVESCSVMHE ALHNHYTQKS LSLSPGAGGG GSGGGGSGGG
GSQDPYVKEA ENLKKYFNAG HSDVADNGTL FLGILKNWKE ESDRKIMQSQ IVSFYFKLEK
NFKDDQSIQK SVETIKEDMN VKFENSNKKK RDDFEKLTNY SVTDLNVQRK AIHELIQVMA
ELSPAAKTGK RKRSQMLERG
SEQ ID NO: 19. IFNγ-Fc
CYCQDPYVKE AENLKKYFNA GHSDVADNGT LFLGILKNWK EESDRKIMQS QIVSFYFKLE
KNFKDDQSIQ KSVETIKEDM NVKFENSNKK KRDDFEKLTN YSVTDLNVQR KAIHELIQVM
AELSPAAKTG KRKRSQMLER GRRASQGGGG SGGGGSGGGG SDKTHTCPPC PAPEAAGGPS
VFLFPPKPKD TLYITREPEV TCVVVDVSHE DPEVKENWYV DGVEVHNAKT KPREEQYNST
YRVVSVLTVL HQDWLNGKEY KCKVSNKALP APIEKTISKA KGQPREPQVY TLPPCREEMT
KNQVSLWCLV KGFYPSDIAV EWESNGQPEN NYKTTPPVLD SDGSFFLYSK LTVDKSRWQQ
GNVFSCSVMH EALHNHYTQK SLSLSPGA
SEQ ID NO: 20. scFv of HuZaf VL-VH-Fc
DIQMTQSPST LSASVGDRVT ITCKASENVD TYVSWYQQKP GKAPKLLIYG ASNRYTGVPS
RFSGSGSGTD FTLTISSLQP DDFATYYCGQ SYNYPFTFGQ GTKVEVKRGG GGSGGGGSGG
GGSQVQLVQS GAELKKPGSS VKVSCKASGY IFTSSWINWV KQAPGQGLEW IGRIDPSDGE
VHYNQDFKDK ATLTVDKSTN TAYMELSSLR SEDTAVYYCA RGFLPWFADW GQGTLVTVSS
GGGGSGGGGS GPLGVRGGGG SGGGGSDKTH TCPPCPAPEA AGGPSVELFP PKPKDTLYIT
REPEVTCVVV DVSHEDPEVK FNWYVDGVEV HNAKTKPREE QYNSTYRVVS VLTVLHQDWL
NGKEYKCKVS NKALPAPIEK TISKAKGQPR EPQVCTLPPS REEMTKNQVS LSCAVKGFYP
SDIAVEWESN GQPENNYKTT PPVLDSDGSF FLVSKLTVDK SRWQQGNVES CSVMHEALHN
HYTQKSLSLS PGK
SEQ ID NO: 21. scFv of HuZaf VH-VL-Fc
QVQLVQSGAE LKKPGSSVKV SCKASGYIFT SSWINWVKQA PGQGLEWIGR IDPSDGEVHY
NQDEKDKATL TVDKSTNTAY MELSSLRSED TAVYYCARGF LPWFADWGRG TLVTVSSGGG
GSGGGGSGGG GSDIQMTQSP STLSASVGDR VTITCKASEN VDTYVSWYQQ KPGKAPKLLI
YGASNRYTGV PSRESGSGSG TDFTLTISSL QPDDFATYYC GQSYNYPFTE GQGTKVEVKR
GGGGSGGGGS GPLGVRGGGG SGGGGSDKTH TCPPCPAPEA AGGPSVELFP PKPKDTLYIT
REPEVTCVVV DVSHEDPEVK FNWYVDGVEV HNAKTKPREE QYNSTYRVVS VLTVLHQDWL
NGKEYKCKVS NKALPAPIEK TISKAKGQPR EPQVCTLPPS REEMTKNQVS LSCAVKGFYP
SDIAVEWESN GQPENNYKTT PPVLDSDGSF FLVSKLTVDK SRWQQGNVFS CSVMHEALHN
HYTQKSLSLS PGK
SEQ ID NO: 22. scFv of AMG811 VL-VH-Fc
EIVLTQSPGT LSLSPGERAT LSCRASQSVS SSYLAWYQQK PGQAPRLLIY GASSRATGIP
DRFSGSGSGT DETLTISRLE PEDFAVYYCQ RSGGSSFTFG PGTKVDIKGG GGSGGGGSGG
GGSEVQLVQS GAEVKKPGES LKISCKGSGY NFTSYWIGWV RQMPGKGLEL MGIIYPGDSD
TRYSPSFQGQ VTISADKSIS TAYLQWSSLK ASDTAMYYCG SGSYFYFDLW GRGTLVTVSS
GGGGSGGGGS GPLGVRGGGG SGGGGSDKTH TCPPCPAPEA AGGPSVELFP PKPKDTLYIT
REPEVTCVVV DVSHEDPEVK FNWYVDGVEV HNAKTKPREE QYNSTYRVVS VLTVLHQDWL
NGKEYKCKVS NKALPAPIEK TISKAKGQPR EPQVCTLPPS REEMTKNQVS LSCAVKGFYP
SDIAVEWESN GQPENNYKTT PPVLDSDGSF FLVSKLTVDK SRWQQGNVES CSVMHEALHN
HYTQKSLSLS PGK
SEQ ID NO: 23. scFv of AMG811 VH-VL-Fc
EVQLVQSGAE VKKPGESLKI SCKGSGYNFT SYWIGWVRQM PGKGLELMGI IYPGDSDTRY
SPSFQGQVTI SADKSISTAY LQWSSLKASD TAMYYCGSGS YFYFDLWGRG TLVTVSSGGG
GSGGGGSGGG GSEIVLTQSP GTLSLSPGER ATLSCRASQS VSSSYLAWYQ QKPGQAPRLL
IYGASSRATG IPDRESGSGS GTDETLTISR LEPEDFAVYY CQRSGGSSFT FGPGTKVDIK
GGGGSGGGGS GPLGVRGGGG SGGGGSDKTH TCPPCPAPEA AGGPSVELFP PKPKDTLYIT
REPEVTCVVV DVSHEDPEVK FNWYVDGVEV HNAKTKPREE QYNSTYRVVS VLTVLHQDWL
NGKEYKCKVS NKALPAPIEK TISKAKGQPR EPQVCTLPPS REEMTKNQVS LSCAVKGFYP
SDIAVEWESN GQPENNYKTT PPVLDSDGSF FLVSKLTVDK SRWQQGNVES CSVMHEALHN
HYTQKSLSLS PGK
SEQ ID NO: 24.
QDPYVKEAEN LKKYFNAGHS DVADNGTLFL GILKNWKEES DRKIMQSQIV SFYFKLEKNE
KDDQSIQKSV ETIKEDMNVK FENSNKKKRD DFEKLTNYSV TDLNVQRKAI HELIQVMAEL
SPAAKTGKRK RSQMLERGGG GSGGGGSGGG GSDKTHTCPP CPAPEAAGGP SVELFPPKPK
DTLYITREPE VTCVVVDVSH EDPEVKENWY VDGVEVHNAK TKPREEQYNS TYRVVSVLTV
LHQDWLNGKE YKCKVSNKAL PAPIEKTISK AKGQPREPQV YTLPPCREEM TKNQVSLWCL
VKGFYPSDIA VEWESNGQPE NNYKTTPPVL DSDGSFFLYS KLTVDKSRWQ QGNVFSCSVM
HEALHNHYTQ KSLSLSPGK
SEQ ID NO: 25.
CYCQDPYVKE AENLKKYFNA GHSDVADNGT LFLGILKNWK EESDRKIMQS QIVSFYFKLE
KNFKDDQSIQ KSVETIKEDM NVKFENSNKK KRDDFEKLTN YSVTDLNVQR KAIHELIQVM
AELSPAAKTG KRKRSQMLER GRRASQGGGG SGGGGSGGGG SDKTHTCPPC PAPEAAGGPS
VFLFPPKPKD TLYITREPEV TCVVVDVSHE DPEVKENWYV DGVEVHNAKT KPREEQYNST
YRVVSVLTVL HQDWLNGKEY KCKVSNKALP APIEKTISKA KGQPREPQVY TLPPCREEMT
KNQVSLWCLV KGFYPSDIAV EWESNGQPEN NYKTTPPVLD SDGSFFLYSK LTVDKSRWQQ
GNVFSCSVMH EALHNHYTQK SLSLSPGK
SEQ ID NO: 26. cleavable peptide linker
GGSLSGRSDN HGS
SEQ ID NO: 27. cleavable linker
GGGGSGGGGS GGGGSISSGL LSSGGSGGSL SGRSDNHGGG GS
SEQ ID NOS: 28-34. MMP-2/MMP-9 Cleavable peptide linkers
GPLGVR (SEQ ID NO: 28)
PLGMWSR (SEQ ID NO: 29)
PLGLWAR (SEQ ID NO: 30)
PQGIAGQR (SEQ ID NO: 31)
PLGLAG (SEQ ID NO: 32)
LALGPR (SEQ ID NO: 33)
GGPLGMLSQS (SEQ ID NO: 34)
SEQ ID NOS: 35-52. urokinase plasminogen activator (uPA) Cleavable
peptide linkers
GGGGRRGGS (SEQ ID NO: 35)
TGRGPSWV (SEQ ID NO: 36)
SARGPSRW (SEQ ID NO: 37)
TARGPSFK (SEQ ID NO: 38)
TARGPSW (SEQ ID NO: 39)
GGWHTGRN (SEQ ID NO: 40)
HTGRSGAL (SEQ ID NO: 41)
PLTGRSGG (SEQ ID NO: 42)
LTGRSGA (SEQ ID NO: 43)
SEQ ID NO: 44. matriptase cleavable peptide linker
RQARVVNG (SEQ ID NO: 44)
SEQ ID NO: 45. matriptase-MMP2/9 dual cleavable peptide linker
VHMPLGFLGP RQARVVNG (SEQ ID NO: 45).
SEQ ID NO: 46. Anti-PD-1 nivolumab HC
EIVLTQSPAT LSLSPGERAT LSCRASQSVS SYLAWYQQKP GQAPRLLIYD ASNRATGIPA
RFSGSGSGTD FTLTISSLEP EDFAVYYCQQ SSNWPRTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC
SEQ ID NO: 47. Anti-PD-1 nivolumab HC - scFv
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVELFPPKP
KDTLMISRTP EVTCVVVDVS QEDPEVQENW YVDGVEVHNA KTKPREEQEN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVESCSV
MHEALHNHYT QKSLSLSLGA GGGGSGGGGS GPLGVRGGGG SGGGGSDIQM TQSPSTLSAS
VGDRVTITCK ASENVDTYVS WYQQKPGKAP KLLIYGASNR YTGVPSRESG SGSGTDETLT
ISSLQPDDFA TYYCGQSYNY PFTFGQGTKV EVKRGGGGSG GGGSGGGGSQ VQLVQSGAEL
KKPGSSVKVS CKASGYIFTS SWINWVKQAP GQGLEWIGRI DPSDGEVHYN QDFKDKATLT
VDKSTNTAYM ELSSLRSEDT AVYYCARGEL PWFADWGQGT LVTVSS
SEQ ID NO: 48. Anti-PD-1 nivolumab HC-ScFv
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVELFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVESCSV
MHEALHNHYT QKSLSLSLGA GGGGSGGGGS GPLGVRGGGG SGGGGSQVQL VQSGAELKKP
GSSVKVSCKA SGYIFTSSWI NWVKQAPGQG LEWIGRIDPS DGEVHYNQDF KDKATLTVDK
STNTAYMELS SLRSEDTAVY YCARGELPWE ADWGRGTLVT VSSGGGGSGG GGSGGGGSDI
QMTQSPSTLS ASVGDRVTIT CKASENVDTY VSWYQQKPGK APKLLIYGAS NRYTGVPSRE
SGSGSGTDFT LTISSLQPDD FATYYCGQSY NYPFTFGQGT KVEVKR
SEQ ID NO: 49. Anti-PD-1 nivolumab HC-ScFv
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEELGG PSVELFPPKP
KDTLMISRTP EVTCVVVDVS QEDPEVQENW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVESCSV
MHEALHNHYT QKSLSLSLGA GGGGSGGGGS GPLGVRGGGG SGGGGSEIVL TQSPGTLSLS
PGERATLSCR ASQSVSSSYL AWYQQKPGQA PRLLIYGASS RATGIPDRES GSGSGTDFTL
TISRLEPEDF AVYYCQRSGG SSFTFGPGTK VDIKGGGGSG GGGSGGGGSE VQLVQSGAEV
KKPGESLKIS CKGSGYNFTS YWIGWVRQMP GKGLELMGII YPGDSDTRYS PSFQGQVTIS
ADKSISTAYL QWSSLKASDT AMYYCGSGSY FYFDLWGRGT LVTVSS
SEQ ID NO: 50. Anti-PD-1 nivolumab HC-scFv
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQEN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVESCSV
MHEALHNHYT QKSLSLSLGA GGGGSGGGGS GPLGVRGGGG SGGGGSEVQL VQSGAEVKKP
GESLKISCKG SGYNFTSYWI GWVRQMPGKG LELMGIIYPG DSDTRYSPSF QGQVTISADK
SISTAYLQWS SLKASDTAMY YCGSGSYFYF DLWGRGTLVT VSSGGGGSGG GGSGGGGSEI
VLTQSPGTLS LSPGERATLS CRASQSVSSS YLAWYQQKPG QAPRLLIYGA SSRATGIPDR
FSGSGSGTDF TLTISRLEPE DFAVYYCQRS GGSSFTFGPG TKVDIK
SEQ ID NO: 51. Anti-PD-1 nivolumab HC-IFNγ
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEELGG PSVELFPPKP
KDTLMISRTP EVTCVVVDVS QEDPEVQENW YVDGVEVHNA KTKPREEQEN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVESCSV
MHEALHNHYT QKSLSLSLGA GGGGSGGGGS GGGGSQDPYV KEAENLKKYF NAGHSDVADN
GTLFLGILKN WKEESDRKIM QSQIVSFYFK LFKNFKDDQS IQKSVETIKE DMNVKFENSN
KKKRDDFEKL TNYSVTDLNV QRKAIHELIQ VMAELSPAAK TGKRKRSQML FRG
SEQ ID NO: 52. Anti-PD-1 nivolumab HC-IFNγ
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVELFPPKP
KDTLMISRTP EVTCVVVDVS QEDPEVQENW YVDGVEVHNA KTKPREEQEN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVESCSV
MHEALHNHYT QKSLSLSLGA GGGGSGGGGS GGGGSCYCQD PYVKEAENLK KYFNAGHSDV
ADNGTLFLGI LKNWKEESDR KIMQSQIVSF YFKLFKNEKD DQSIQKSVET IKEDMNVKFF
NSNKKKRDDF EKLTNYSVTD LNVQRKAIHE LIQVMAELSP AAKTGKRKRS QMLERGRRAS
Q
SEQ ID NO: 53. Anti-PD-1 pembrolizumab LC
EIVLTQSPAT LSLSPGERAT LSCRASKGVS TSGYSYLHWY QQKPGQAPRL LIYLASYLES
GVPARFSGSG SGTDETLTIS SLEPEDFAVY YCQHSRDLPL TFGGGTKVEI KRTVAAPSVF
IFPPSDEQLK SGTASVVCLL NNFYPREAKV QWKVDNALQS GNSQESVTEQ DSKDSTYSLS
STLTLSKADY EKHKVYACEV THQGLSSPVT KSENRGEC
SEQ ID NO: 54. Anti-PD-1 pembrolizumab HC-scFv
QVQLVQSGVE VKKPGASVKV SCKASGYTFT NYYMYWVRQA PGQGLEWMGG INPSNGGTNF
NEKFKNRVTL TTDSSTTTAY MELKSLQFDD TAVYYCARRD YRFDMGEDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGPL GVRGGGGSGG GGSDIQMTQS
PSTLSASVGD RVTITCKASE NVDTYVSWYQ QKPGKAPKLL IYGASNRYTG VPSRESGSGS
GTDFTLTISS LQPDDFATYY CGQSYNYPFT FGQGTKVEVK RGGGGSGGGG SGGGGSQVQL
VQSGAELKKP GSSVKVSCKA SGYIFTSSWI NWVKQAPGQG LEWIGRIDPS DGEVHYNQDE
KDKATLTVDK STNTAYMELS SLRSEDTAVY YCARGELPWF ADWGQGTLVT VSS
SEQ ID NO: 55. Anti-PD-1 Pembrolizumab HC-scFv
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YRFDMGEDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQENWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGPL GVRGGGGSGG GGSQVQLVQS
GAELKKPGSS VKVSCKASGY IFTSSWINWV KQAPGQGLEW IGRIDPSDGE VHYNQDEKDK
ATLTVDKSTN TAYMELSSLR SEDTAVYYCA RGFLPWFADW GRGTLVTVSS GGGGSGGGGS
GGGGSDIQMT QSPSTLSASV GDRVTITCKA SENVDTYVSW YQQKPGKAPK LLIYGASNRY
TGVPSRESGS GSGTDFTLTI SSLQPDDFAT YYCGQSYNYP FTFGQGTKVE VKR
SEQ ID NO: 56. Anti-PD-1 pembrolizumab HC-scFv
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YREDMGFDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGPL GVRGGGGSGG GGSEIVLTQS
PGTLSLSPGE RATLSCRASQ SVSSSYLAWY QQKPGQAPRL LIYGASSRAT GIPDRESGSG
SGTDETLTIS RLEPEDFAVY YCQRSGGSSF TFGPGTKVDI KGGGGSGGGG SGGGGSEVQL
VQSGAEVKKP GESLKISCKG SGYNFTSYWI GWVRQMPGKG LELMGIIYPG DSDTRYSPSE
QGQVTISADK SISTAYLQWS SLKASDTAMY YCGSGSYFYF DLWGRGTLVT VSS
SEQ ID NO: 57. Anti-PD-1 pembrolizumab HC-scFv
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YREDMGEDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGPL GVRGGGGSGG GGSEVQLVQS
GAEVKKPGES LKISCKGSGY NFTSYWIGWV RQMPGKGLEL MGIIYPGDSD TRYSPSFQGQ
VTISADKSIS TAYLQWSSLK ASDTAMYYCG SGSYFYFDLW GRGTLVTVSS GGGGSGGGGS
GGGGSEIVLT QSPGTLSLSP GERATLSCRA SQSVSSSYLA WYQQKPGQAP RLLIYGASSR
ATGIPDRESG SGSGTDETLT ISRLEPEDFA VYYCQRSGGS SFTFGPGTKV DIK
SEQ ID NO: 58. Anti-PD-1 pembrolizumab HC-IFNγ
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YREDMGEDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGGG GSQDPYVKEA ENLKKYFNAG
HSDVADNGTL FLGILKNWKE ESDRKIMQSQ IVSFYFKLEK NEKDDQSIQK SVETIKEDMN
VKFFNSNKKK RDDFEKLTNY SVTDLNVQRK AIHELIQVMA ELSPAAKTGK RKRSQMLERG
SEQ ID NO: 59. Anti-PD-1 pembrolizumab HC-IFNγ
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YREDMGEDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGGG GSCYCQDPYV KEAENLKKYF
NAGHSDVADN GTLFLGILKN WKEESDRKIM QSQIVSFYFK LFKNEKDDQS IQKSVETIKE
DMNVKFFNSN KKKRDDFEKL TNYSVTDLNV QRKAIHELIQ VMAELSPAAK TGKRKRSQML
FRGRRASQ
SEQ ID NO: 60. scFv1 of sifalimumab VL-VH
EIVLTQSPGT LSLSPGERAT LSCRASQSVS STYLAWYQQK PGQAPRLLIY GASSRATGIP
DRESGSGSGT DETLTISRLE PEDFAVYYCQ QYGSSPRTFG QGTKVEIKGG GGSGGGGSGG
GGSQVQLVQS GAEVKKPGAS VKVSCKASGY TFTSYSISWV RQAPGQGLEW MGWISVYNGN
TNYAQKFQGR VTMTTDTSTS TAYLELRSLR SDDTAVYYCA RDPIAAGYWG QGTLVTVSS
SEQ ID NO: 61. scFv2 of sifalimumab VH-VL
EIVLTQSPGT LSLSPGERAT LSCRASQSVS STYLAWYQQK PGQAPRLLIY GASSRATGIP
DRFSGSGSGT DFTLTISRLE PEDFAVYYCQ QYGSSPRTFG QGTKVEIKGG GGSGGGGSGG
GGSDIQMTQS PSSLSASVGD RVTITCRASQ SVSTSSYSYM HWYQQKPGKA PKVLISYASN
LESGVPSRES GSGSGTDETL TISSLQPEDF ATYYCQHSWG IPRTFGQGTK VEIK
SEQ ID NO: 62. rontalizumab-VL
QVQLVQSGAE VKKPGASVKV SCKASGYTFT SYSISWVRQA PGQGLEWMGW ISVYNGNTNY
AQKFQGRVTM TTDTSTSTAY LELRSLRSDD TAVYYCARDP IAAGYWGQGT LVTVSS
SEQ ID NO: 63. VL chain of an antibody against IFN-alpha
QSVLTQPPSV SGAPGQRVTI SCSGSSSNIG SNYVSWYQQL PGTAPKLLIY DNNQRPSGVP
DRESGSKSGT SASLAITGLQ SEDEADYYCQ SNDASLVEVE GGGTKLTVLG QP
SEQ ID NO: 64. VH of an antibody against IFN-alpha
EVQLVESGGG LVQPGGSLRL SCAASGFTES SYAMSWVRQA PGKGLEWVSA ISGSGGSTYY
ADSVKGRFTI SRDNSKNTLY LQMNSLRAED TAVYYCARYY SFYTSFDYWG QGTLVTVSS
SEQ ID NO: 65. IFNα 2b
CDLPQTHSLG SRRTLMLLAQ MRKISLESCL KDRHDFGFPQ EEFGNQFQKA ETIPVLHEMI
QQIFNLESTK DSSAAWDETL LDKFYTELYQ QLNDLEACVI QGVGVTETPL MKEDSILAVR
KYFQRITLYL KEKKYSPCAW EVVRAEIMRS FSLSTNLQES LRSKE
SEQ ID NO: 66. IFNα 2a
CDLPQTHSLG SRRTLMLLAQ MRKISLESCL KDRHDFGFPQ EEFGNQFQKA ETIPVLHEMI
QQIFNLESTK DSSAAWDETL LDKFYTELYQ QLNDLEACVI QGVGVTETPL MKEDSILAVR
KYFQRITLYL KEKKYSPCAW EVVRAEIMRS FSLSTNLQES LRSKE
SEQ ID NO: 67. Fc-scFv1-sifalimumab
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVCT LPPSREEMTK NQVSLSCAVK GFYPSDIAVE WESNGQPENN YKTTPPVLDS
DGSFFLVSKL TVDKSRWQQG NVFSCSVMHE ALHNHYTQKS LSLSPGAGGG GSGGGGSGPL
GVRGGGGSGG GGSEIVLTQS PGTLSLSPGE RATLSCRASQ SVSSTYLAWY QQKPGQAPRL
LIYGASSRAT GIPDRESGSG SGTDETLTIS RLEPEDFAVY YCQQYGSSPR TFGQGTKVEI
KGGGGSGGGG SGGGGSQVQL VQSGAEVKKP GASVKVSCKA SGYTFTSYSI SWVRQAPGQG
LEWMGWISVY NGNTNYAQKF QGRVTMTTDT STSTAYLELR SLRSDDTAVY YCARDPIAAG
YWGQGTLVTV SS
SEQ ID NO: 68. Fc-scFv1- sifalimumab
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVCT LPPSREEMTK NQVSLSCAVK GFYPSDIAVE WESNGQPENN YKTTPPVLDS
DGSFFLVSKL TVDKSRWQQG NVESCSVMHE ALHNHYTQKS LSLSPGAGGG GSVHMPLGEL
GPRQARVVNA GGGGSGGGGS EIVLTQSPGT LSLSPGERAT LSCRASQSVS STYLAWYQQK
PGQAPRLLIY GASSRATGIP DRESGSGSGT DETLTISRLE PEDFAVYYCQ QYGSSPRTFG
QGTKVEIKGG GGSGGGGSGG GGSQVQLVQS GAEVKKPGAS VKVSCKASGY TFTSYSISWV
RQAPGQGLEW MGWISVYNGN TNYAQKFQGR VTMTTDTSTS TAYLELRSLR SDDTAVYYCA
RDPIAAGYWG QGTLVTVSS
SEQ ID NO: 69. Fc-scFv2 sifalimumab
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVCT LPPSREEMTK NQVSLSCAVK GFYPSDIAVE WESNGQPENN YKTTPPVLDS
DGSFFLVSKL TVDKSRWQQG NVESCSVMHE ALHNHYTQKS LSLSPGAGGG GSGGGGSGPL
GVRGGGGSGG GGSEIVLTQS PGTLSLSPGE RATLSCRASQ SVSSTYLAWY QQKPGQAPRL
LIYGASSRAT GIPDRESGSG SGTDETLTIS RLEPEDFAVY YCQQYGSSPR TFGQGTKVEI
KGGGGSGGGG SGGGGSDIQM TQSPSSLSAS VGDRVTITCR ASQSVSTSSY SYMHWYQQKP
GKAPKVLISY ASNLESGVPS RESGSGSGTD FTLTISSLQP EDFATYYCQH SWGIPRTFGQ
GTKVEIK
SEQ ID NO: 70. Fc-scFv2 sifalimumab
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVCT LPPSREEMTK NQVSLSCAVK GFYPSDIAVE WESNGQPENN YKTTPPVLDS
DGSFFLVSKL TVDKSRWQQG NVESCSVMHE ALHNHYTQKS LSLSPGAGGG GSVHMPLGEL
GPRQARVVNA GGGGSGGGGS EIVLTQSPGT LSLSPGERAT LSCRASQSVS STYLAWYQQK
PGQAPRLLIY GASSRATGIP DRESGSGSGT DETLTISRLE PEDFAVYYCQ QYGSSPRTFG
QGTKVEIKGG GGSGGGGSGG GGSDIQMTQS PSSLSASVGD RVTITCRASQ SVSTSSYSYM
HWYQQKPGKA PKVLISYASN LESGVPSRES GSGSGTDFTL TISSLQPEDF ATYYCQHSWG
IPRTFGQGTK VEIK
SEQ ID NO: 71. Fc-IFNα-2b
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVYT LPPCREEMTK NQVSLWCLVK GFYPSDIAVE WESNGQPENN YKTTPPVLDS
DGSFFLYSKL TVDKSRWQQG NVESCSVMHE ALHNHYTQKS LSLSPGAGGG GSGGGGSGGG
GSCDLPQTHS LGSRRTLMLL AQMRKISLES CLKDRHDFGF PQEEFGNQFQ KAETIPVLHE
MIQQIFNLES TKDSSAAWDE TLLDKFYTEL YQQLNDLEAC VIQGVGVTET PLMKEDSILA
VRKYFQRITL YLKEKKYSPC AWEVVRAEIM RSFSLSTNLQ ESLRSKE
SEQ ID NO: 72. Fc IFNα-2a
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVYT LPPCREEMTK NQVSLWCLVK GFYPSDIAVE WESNGQPENN YKTTPPVLDS
DGSFFLYSKL TVDKSRWQQG NVFSCSVMHE ALHNHYTQKS LSLSPGAGGG GSGGGGSGGG
GSCDLPQTHS LGSRRTLMLL AQMRKISLES CLKDRHDFGF PQEEFGNQFQ KAETIPVLHE
MIQQIFNLES TKDSSAAWDE TLLDKFYTEL YQQLNDLEAC VIQGVGVTET PLMKEDSILA
VRKYFQRITL YLKEKKYSPC AWEVVRAEIM RSFSLSTNLQ ESLRSKE
SEQ ID NO: 73. scFv1 of sifalimumab VL-VH-Fc
EIVLTQSPGT LSLSPGERAT LSCRASQSVS STYLAWYQQK PGQAPRLLIY GASSRATGIP
DRESGSGSGT DFTLTISRLE PEDFAVYYCQ QYGSSPRTFG QGTKVEIKGG GGSGGGGSGG
GGSQVQLVQS GAEVKKPGAS VKVSCKASGY TFTSYSISWV RQAPGQGLEW MGWISVYNGN
TNYAQKFQGR VTMTTDTSTS TAYLELRSLR SDDTAVYYCA RDPIAAGYWG QGTLVTVSSG
GGGSGGGGSG PLGVRGGGGS GGGGSDKTHT CPPCPAPEAA GGPSVELFPP KPKDTLYITR
EPEVTCVVVD VSHEDPEVKF NWYVDGVEVH NAKTKPREEQ YNSTYRVVSV LTVLHQDWLN
GKEYKCKVSN KALPAPIEKT ISKAKGQPRE PQVCTLPPSR EEMTKNQVSL SCAVKGFYPS
DIAVEWESNG QPENNYKTTP PVLDSDGSFF LVSKLTVDKS RWQQGNVESC SVMHEALHNH
YTQKSLSLSP GK
SEQ ID NO: 74. scFv1 of sifalimumab VH-VL-Fc
EIVLTQSPGT LSLSPGERAT LSCRASQSVS STYLAWYQQK PGQAPRLLIY GASSRATGIP
DRESGSGSGT DETLTISRLE PEDFAVYYCQ QYGSSPRTFG QGTKVEIKGG GGSGGGGSGG
GGSQVQLVQS GAEVKKPGAS VKVSCKASGY TFTSYSISWV RQAPGQGLEW MGWISVYNGN
TNYAQKFQGR VTMTTDTSTS TAYLELRSLR SDDTAVYYCA RDPIAAGYWG QGTLVTVSSG
GGGSVHMPLG FLGPRQARVV NAGGGGSGGG GSDKTHTCPP CPAPEAAGGP SVELFPPKPK
DTLYITREPE VTCVVVDVSH EDPEVKENWY VDGVEVHNAK TKPREEQYNS TYRVVSVLTV
LHQDWLNGKE YKCKVSNKAL PAPIEKTISK AKGQPREPQV CTLPPSREEM TKNQVSLSCA
VKGFYPSDIA VEWESNGQPE NNYKTTPPVL DSDGSFFLVS KLTVDKSRWQ QGNVFSCSVM
HEALHNHYTQ KSLSLSPGK
SEQ ID NO: 75. scFv2 of VL-VH-Fc
EIVLTQSPGT LSLSPGERAT LSCRASQSVS STYLAWYQQK PGQAPRLLIY GASSRATGIP
DRESGSGSGT DFTLTISRLE PEDFAVYYCQ QYGSSPRTFG QGTKVEIKGG GGSGGGGSGG
GGSDIQMTQS PSSLSASVGD RVTITCRASQ SVSTSSYSYM HWYQQKPGKA PKVLISYASN
LESGVPSRES GSGSGTDETL TISSLQPEDF ATYYCQHSWG IPRTFGQGTK VEIKGGGGSG
GGGSGPLGVR GGGGSGGGGS DKTHTCPPCP APEAAGGPSV FLFPPKPKDT LYITREPEVT
CVVVDVSHED PEVKENWYVD GVEVHNAKTK PREEQYNSTY RVVSVLTVLH QDWLNGKEYK
CKVSNKALPA PIEKTISKAK GQPREPQVCT LPPSREEMTK NQVSLSCAVK GFYPSDIAVE
WESNGQPENN YKTTPPVLDS DGSFFLVSKL TVDKSRWQQG NVESCSVMHE ALHNHYTQKS
LSLSPGK
SEQ ID NO: 76. scFv 2 of sifalimumab VH-VL-Fc
EIVLTQSPGT LSLSPGERAT LSCRASQSVS STYLAWYQQK PGQAPRLLIY GASSRATGIP
DRFSGSGSGT DETLTISRLE PEDFAVYYCQ QYGSSPRTFG QGTKVEIKGG GGSGGGGSGG
GGSDIQMTQS PSSLSASVGD RVTITCRASQ SVSTSSYSYM HWYQQKPGKA PKVLISYASN
LESGVPSRES GSGSGTDFTL TISSLQPEDF ATYYCQHSWG IPRTFGQGTK VEIKGGGGSV
HMPLGFLGPR QARVVNAGGG GSGGGGSDKT HTCPPCPAPE AAGGPSVELF PPKPKDTLYI
TREPEVTCVV VDVSHEDPEV KENWYVDGVE VHNAKTKPRE EQYNSTYRVV SVLTVLHQDW
LNGKEYKCKV SNKALPAPIE KTISKAKGQP REPQVCTLPP SREEMTKNQV SLSCAVKGFY
PSDIAVEWES NGQPENNYKT TPPVLDSDGS FFLVSKLTVD KSRWQQGNVF SCSVMHEALH
NHYTQKSLSL SPGK
SEQ ID NO: 77. IFNα-2b-Fc
CDLPQTHSLG SRRTLMLLAQ MRKISLESCL KDRHDFGFPQ EEFGNQFQKA ETIPVLHEMI
QQIFNLESTK DSSAAWDETL LDKFYTELYQ QLNDLEACVI QGVGVTETPL MKEDSILAVR
KYFQRITLYL KEKKYSPCAW EVVRAEIMRS FSLSTNLQES LRSKEGGGGS GGGGSGGGGS
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVYT LPPCREEMTK NQVSLWCLVK GFYPSDIAVE WESNGQPENN YKTTPPVLDS
DGSFFLYSKL TVDKSRWQQG NVESCSVMHE ALHNHYTQKS LSLSPGK
SEQ ID NO: 78. IFNα-2a-Fc
CDLPQTHSLG SRRTLMLLAQ MRKISLESCL KDRHDFGFPQ EEFGNQFQKA ETIPVLHEMI
QQIFNLESTK DSSAAWDETL LDKFYTELYQ QLNDLEACVI QGVGVTETPL MKEDSILAVR
KYFQRITLYL KEKKYSPCAW EVVRAEIMRS FSLSTNLQES LRSKEGGGGS GGGGSGGGGS
DKTHTCPPCP APEAAGGPSV FLFPPKPKDT LYITREPEVT CVVVDVSHED PEVKENWYVD
GVEVHNAKTK PREEQYNSTY RVVSVLTVLH QDWLNGKEYK CKVSNKALPA PIEKTISKAK
GQPREPQVYT LPPCREEMTK NQVSLWCLVK GFYPSDIAVE WESNGQPENN YKTTPPVLDS
DGSFFLYSKL TVDKSRWQQG NVESCSVMHE ALHNHYTQKS LSLSPGK
SEQ ID NO: 79. Anti-PD-1 nivolumab HC-scFv1
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVELFPPKP
KDTLMISRTP EVTCVVVDVS QEDPEVQENW YVDGVEVHNA KTKPREEQEN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVESCSV
MHEALHNHYT QKSLSLSLGA GGGGSGGGGS GPLGVRGGGG SGGGGSEIVL TQSPGTLSLS
PGERATLSCR ASQSVSSTYL AWYQQKPGQA PRLLIYGASS RATGIPDRES GSGSGTDETL
TISRLEPEDF AVYYCQQYGS SPRTFGQGTK VEIKGGGGSG GGGSGGGGSQ VQLVQSGAEV
KKPGASVKVS CKASGYTFTS YSISWVRQAP GQGLEWMGWI SVYNGNTNYA QKFQGRVTMT
TDTSTSTAYL ELRSLRSDDT AVYYCARDPI AAGYWGQGTL VTVSS
SEQ ID NO: 80. Anti-PD-1 nivolumab HC-scFv1
QVQLVESGGG VVQPGRSLRL DCKASGITES NSGMHWVRQA PGKGLEWVAV IWYDGSKRYY
ADSVKGRETI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVELFPPKP
KDTLMISRTP EVTCVVVDVS QEDPEVQENW YVDGVEVHNA KTKPREEQEN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLGA GGGGSGGGGS GGGGSEIVLT QSPGTLSLSP GERATLSCRA
SQSVSSTYLA WYQQKPGQAP RLLIYGASSR ATGIPDRESG SGSGTDFTLT ISRLEPEDFA
VYYCQQYGSS PRTFGQGTKV EIKGGGGSGG GGSGGGGSQV QLVQSGAEVK KPGASVKVSC
KASGYTFTSY SISWVRQAPG QGLEWMGWIS VYNGNTNYAQ KFQGRVTMTT DTSTSTAYLE
LRSLRSDDTA VYYCARDPIA AGYWGQGTLV TVSS
SEQ ID NO: 81. Anti-PD-1 nivolumab HC-scFv2
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVELFPPKP
KDTLMISRTP EVTCVVVDVS QEDPEVQENW YVDGVEVHNA KTKPREEQEN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLGA GGGGSGGGGS GPLGVRGGGG SGGGGSEIVL TQSPGTLSLS
PGERATLSCR ASQSVSSTYL AWYQQKPGQA PRLLIYGASS RATGIPDRES GSGSGTDETL
TISRLEPEDF AVYYCQQYGS SPRTFGQGTK VEIKGGGGSG GGGSGGGGSD IQMTQSPSSL
SASVGDRVTI TCRASQSVST SSYSYMHWYQ QKPGKAPKVL ISYASNLESG VPSRESGSGS
GTDFTLTISS LQPEDFATYY CQHSWGIPRT FGQGTKVEIK
SEQ ID NO: 82. Anti-PD-1 nivolumab HC-scFv
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVELFPPKP
KDTLMISRTP EVTCVVVDVS QEDPEVQENW YVDGVEVHNA KTKPREEQEN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVESCSV
MHEALHNHYT QKSLSLSLGA GGGGSGGGGS GGGGSEIVLT QSPGTLSLSP GERATLSCRA
SQSVSSTYLA WYQQKPGQAP RLLIYGASSR ATGIPDRESG SGSGTDETLT ISRLEPEDFA
VYYCQQYGSS PRTFGQGTKV EIKGGGGSGG GGSGGGGSDI QMTQSPSSLS ASVGDRVTIT
CRASQSVSTS SYSYMHWYQQ KPGKAPKVLI SYASNLESGV PSRESGSGSG TDFTLTISSL
QPEDFATYYC QHSWGIPRTF GQGTKVEIK
SEQ ID NO: 83. Anti-PD-1 nivolumab HC-IFNα-2b
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQEN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVESCSV
MHEALHNHYT QKSLSLSLGA GGGGSGGGGS GGGGSCDLPQ THSLGSRRTL MLLAQMRKIS
LFSCLKDRHD FGFPQEEFGN QFQKAETIPV LHEMIQQIFN LFSTKDSSAA WDETLLDKFY
TELYQQLNDL EACVIQGVGV TETPLMKEDS ILAVRKYFQR ITLYLKEKKY SPCAWEVVRA
EIMRSFSLST NLQESLRSKE
SEQ ID NO: 84. Anti-PD-1 nivolumab HC-IFNα-2a
QVQLVESGGG VVQPGRSLRL DCKASGITES NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVELFPPKP
KDTLMISRTP EVTCVVVDVS QEDPEVQENW YVDGVEVHNA KTKPREEQEN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVESCSV
MHEALHNHYT QKSLSLSLGA GGGGSGGGGS GGGGSCDLPQ THSLGSRRTL MLLAQMRKIS
LFSCLKDRHD FGFPQEEFGN QFQKAETIPV LHEMIQQIFN LFSTKDSSAA WDETLLDKFY
TELYQQLNDL EACVIQGVGV TETPLMKEDS ILAVRKYFQR ITLYLKEKKY SPCAWEVVRA
EIMRSFSLST NLQESLRSKE
SEQ ID NO: 85. Anti-PD-1 pembrolizumab HC-scFv1
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YRFDMGEDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGPL GVRGGGGSGG GGSEIVLTQS
PGTLSLSPGE RATLSCRASQ SVSSTYLAWY QQKPGQAPRL LIYGASSRAT GIPDRESGSG
SGTDFTLTIS RLEPEDFAVY YCQQYGSSPR TFGQGTKVEI KGGGGSGGGG SGGGGSQVQL
VQSGAEVKKP GASVKVSCKA SGYTFTSYSI SWVRQAPGQG LEWMGWISVY NGNTNYAQKE
QGRVTMTTDT STSTAYLELR SLRSDDTAVY YCARDPIAAG YWGQGTLVTV SS
SEQ ID NO: 86. Anti-PD-1 pembrolizumab HC-scFv1
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YREDMGFDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWINGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGGG GSEIVLTQSP GTLSLSPGER
ATLSCRASQS VSSTYLAWYQ QKPGQAPRLL IYGASSRATG IPDRESGSGS GTDETLTISR
LEPEDFAVYY CQQYGSSPRT FGQGTKVEIK GGGGSGGGGS GGGGSQVQLV QSGAEVKKPG
ASVKVSCKAS GYTFTSYSIS WVRQAPGQGL EWMGWISVYN GNTNYAQKFQ GRVTMTTDTS
TSTAYLELRS LRSDDTAVYY CARDPIAAGY WGQGTLVTVS S
SEQ ID NO: 87. Anti-PD-1 pembrolizumab HC-scFv2
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YREDMGEDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGPL GVRGGGGSGG GGSEIVLTQS
PGTLSLSPGE RATLSCRASQ SVSSTYLAWY QQKPGQAPRL LIYGASSRAT GIPDRESGSG
SGTDFTLTIS RLEPEDFAVY YCQQYGSSPR TFGQGTKVEI KGGGGSGGGG SGGGGSDIQM
TQSPSSLSAS VGDRVTITCR ASQSVSTSSY SYMHWYQQKP GKAPKVLISY ASNLESGVPS
RFSGSGSGTD FTLTISSLQP EDFATYYCQH SWGIPRTFGQ GTKVEIK
SEQ ID NO: 88. Anti-PD-1 pembrolizumab HC-scFv
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YREDMGFDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGGG GSEIVLTQSP GTLSLSPGER
ATLSCRASQS VSSTYLAWYQ QKPGQAPRLL IYGASSRATG IPDRESGSGS GTDFTLTISR
LEPEDFAVYY CQQYGSSPRT FGQGTKVEIK GGGGSGGGGS GGGGSDIQMT QSPSSLSASV
GDRVTITCRA SQSVSTSSYS YMHWYQQKPG KAPKVLISYA SNLESGVPSR FSGSGSGTDE
TLTISSLQPE DFATYYCQHS WGIPRTFGQG TKVEIK
SEQ ID NO: 89. Anti-PD-1 pembrolizumab HC-IFNα-2b
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YREDMGFDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGGG GSCDLPQTHS LGSRRTLMLL
AQMRKISLFS CLKDRHDFGF PQEEFGNQFQ KAETIPVLHE MIQQIENLES TKDSSAAWDE
TLLDKFYTEL YQQLNDLEAC VIQGVGVTET PLMKEDSILA VRKYFQRITL YLKEKKYSPC
AWEVVRAEIM RSFSLSTNLQ ESLRSKE
SEQ ID NO: 90. Anti-PD-1 pembrolizumab HC-IFNα-2a
QVQLVQSGVE VKKPGASVKV SCKASGYTFT NYYMYWVRQA PGQGLEWMGG INPSNGGTNE
NEKFKNRVTL TTDSSTTTAY MELKSLQFDD TAVYYCARRD YREDMGFDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQENSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGAGGG GSGGGGSGGG GSCDLPQTHS LGSRRTLMLL
AQMRKISLES CLKDRHDFGF PQEEFGNQFQ KAETIPVLHE MIQQIFNLES TKDSSAAWDE
TLLDKFYTEL YQQLNDLEAC VIQGVGVTET PLMKEDSILA VRKYFQRITL YLKEKKYSPC
AWEVVRAEIM RSFSLSTNLQ ESLRSKE
SEQ ID NO: 91. Anti-CS1 antibody elotuzumab heavy chain
EVQLVESGGG LVQPGGSLRL SCAASGEDES RYWMSWVRQA PGKGLEWIGE INPDSSTINY
APSLKDKFII SRDNAKNSLY LQMNSLRAED TAVYYCARPD GNYWYFDVWG QGTLVTVSSA
STKGPSVFPL APSSKSTSGG TAALGCLVKD YFPEPVTVSW NSGALTSGVH TEPAVLQSSG
LYSLSSVVTV PSSSLGTQTY ICNVNHKPSN TKVDKKVEPK SCDKTHTCPP CPAPELLGGP
SVFLFPPKPK DTLMISRTPE VTCVVVDVSH EDPEVKENWY VDGVEVHNAK TKPREEQYNS
TYRVVSVLTV LHQDWLNGKE YKCKVSNKAL PAPIEKTISK AKGQPREPQV YTLPPSRDEL
TKNQVSLTCL VKGFYPSDIA VEWESNGQPE NNYKTTPPVL DSDGSFFLYS KLTVDKSRWQ
QGNVFSCSVM HEALHNHYTQ KSLSLSPGK
SEQ ID NO: 92. Anti-CS1 antibody elotuzumab light chain
DIQMTQSPSS LSASVGDRVT ITCKASQDVG IAVAWYQQKP GKVPKLLIYW ASTRHTGVPD
RFSGSGSGTD FTLTISSLQP EDVATYYCQQ YSSYPYTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC
SEQ ID NO: 93. Anti-CD38 antibody daratumumab heavy chain
EVQLLESGGG LVQPGGSLRL SCAVSGFTEN SFAMSWVRQA PGKGLEWVSA ISGSGGGTYY
ADSVKGRFTI SRDNSKNTLY LQMNSLRAED TAVYFCAKDK ILWFGEPVED YWGQGTLVTV
SSASTKGPSV FPLAPSSKST SGGTAALGCL VKDYFPEPVT VSWNSGALTS GVHTFPAVLQ
SSGLYSLSSV VTVPSSSLGT QTYICNVNHK PSNTKVDKRV EPKSCDKTHT CPPCPAPELL
GGPSVELFPP KPKDTLMISR TPEVTCVVVD VSHEDPEVKF NWYVDGVEVH NAKTKPREEQ
YNSTYRVVSV LTVLHQDWLN GKEYKCKVSN KALPAPIEKT ISKAKGQPRE PQVYTLPPSR
EEMTKNQVSL TCLVKGFYPS DIAVEWESNG QPENNYKTTP PVLDSDGSFF LYSKLTVDKS
RWQQGNVFSC SVMHEALHNH YTQKSLSLSP GK
SEQ ID NO: 94. Anti-CD38 antibody daratumumab light chain
EIVLTQSPAT LSLSPGERAT LSCRASQSVS SYLAWYQQKP GQAPRLLIYD ASNRATGIPA
RFSGSGSGTD FTLTISSLEP EDFAVYYCQQ RSNWPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC
SEQ ID NO: 95. Anti-CD38 antibody isatuximab heavy chain
QVQLVQSGAE VAKPGTSVKL SCKASGYTFT DYWMQWVKQR PGQGLEWIGT IYPGDGDTGY
AQKFQGKATL TADKSSKTVY MHLSSLASED SAVYYCARGD YYGSNSLDYW GQGTSVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVFLFPPKP KDTLMISRTP EVTCVVVDVS HEDPEVKENW YVDGVEVHNA KTKPREEQYN
STYRVVSVLT VLHQDWLNGK EYKCKVSNKA LPAPIEKTIS KAKGQPREPQ VYTLPPSRDE
LTKNQVSLTC LVKGFYPSDI AVEWESNGQP ENNYKTTPPV LDSDGSFFLY SKLTVDKSRW
QQGNVFSCSV MHEALHNHYT QKSLSLSPGK
SEQ ID NO: 96. Anti-CD38 antibody isatuximab light chain
DIVMTQSHLS MSTSLGDPVS ITCKASQDVS TVVAWYQQKP GQSPRRLIYS ASYRYIGVPD
RFTGSGAGTD FTFTISSVQA EDLAVYYCQQ HYSPPYTFGG GTKLEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC
SEQ ID NO: 97. Anti-CS1 antibody elotuzumab heavy chain variable
domain
EVQLVESGGG LVQPGGSLRL SCAASGEDES RYWMSWVRQA PGKGLEWIGE INPDSSTINY
APSLKDKFII SRDNAKNSLY LQMNSLRAED TAVYYCARPD GNYWYFDVWG QGTLVTVSS
SEQ ID NO: 98. Anti-CS1 antibody elotuzumab light chain variable
domain
DIQMTQSPSS LSASVGDRVT ITCKASQDVG IAVAWYQQKP GKVPKLLIYW ASTRHTGVPD
RFSGSGSGTD FTLTISSLQP EDVATYYCQQ YSSYPYTFGQ GTKVEIK
SEQ ID NO: 99. Anti-CD38 antibody daratumumab heavy chain variable
domain
EVQLLESGGG LVQPGGSLRL SCAVSGFTEN SFAMSWVRQA PGKGLEWVSA ISGSGGGTYY
ADSVKGRETI SRDNSKNTLY LQMNSLRAED TAVYFCAKDK ILWFGEPVED YWGQGTLVTV
SS
SEQ ID NO: 100. Anti-CD38 antibody daratumumab light chain variable
domain
EIVLTQSPAT LSLSPGERAT LSCRASQSVS SYLAWYQQKP GQAPRLLIYD ASNRATGIPA
RFSGSGSGTD FTLTISSLEP EDFAVYYCQQ RSNWPPTFGQ GTKVEIK
SEQ ID NO: 101. Anti-CD38 antibody isatuximab Heavy Chain variable
domain
QVQLVQSGAE VAKPGTSVKL SCKASGYTFT DYWMQWVKQR PGQGLEWIGT IYPGDGDTGY
AQKFQGKATL TADKSSKTVY MHLSSLASED SAVYYCARGD YYGSNSLDYW GQGTSVTVSS
SEQ ID NO: 102. Anti-CD38 antibody isatuximab light chain variable
domain
DIVMTQSHLS MSTSLGDPVS ITCKASQDVS TVVAWYQQKP GQSPRRLIYS ASYRYIGVPD
RFTGSGAGTD FTFTISSVQA EDLAVYYCQQ HYSPPYTFGG GTKLEIK
SEQ ID NO: 103. felzartamab heavy chain variable domain
QVQLVESGGG LVQPGGSLRL SCAASGFTFS SYYMNWVRQA PGKGLEWVSG ISGDPSNTYY
ADSVKGRFTI SRDNSKNTLY LQMNSLRAED TAVYYCARDL PLVYTGFAYW GQGTLVTVSS
SEQ ID NO: 104. felzartamab Light chain variable domain
DIELTQPPSV SVAPGQTARI SCSGDNLRHY YVYWYQQKPG QAPVLVIYGD SKRPSGIPER
FSGSNSGNTA TLTISGTQAE DEADYYCQTY TGGASLVEGG GTKLTVL
SEQ ID NO: 105. felzartamab heavy chain
QVQLVESGGG LVQPGGSLRL SCAASGFTES SYYMNWVRQA PGKGLEWVSG ISGDPSNTYY
ADSVKGRFTI SRDNSKNTLY LQMNSLRAED TAVYYCARDL PLVYTGFAYW GQGTLVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKRVEP KSCDKTHTCP PCPAPELLGG
PSVELFPPKP KDTLMISRTP EVTCVVVDVS HEDPEVKENW YVDGVEVHNA KTKPREEQYN
STYRVVSVLT VLHQDWLNGK EYKCKVSNKA LPAPIEKTIS KAKGQPREPQ VYTLPPSREE
MTKNQVSLTC LVKGFYPSDI AVEWESNGQP ENNYKTTPPV LDSDGSFFLY SKLTVDKSRW
QQGNVFSCSV MHEALHNHYT QKSLSLSPGK
SEQ ID NO: 106. felzartamab light chain
DIELTQPPSV SVAPGQTARI SCSGDNLRHY YVYWYQQKPG QAPVLVIYGD SKRPSGIPER
FSGSNSGNTA TLTISGTQAE DEADYYCQTY TGGASLVEGG GTKLTVLGQP KAAPSVTLEP
PSSEELQANK ATLVCLISDF YPGAVTVAWK ADSSPVKAGV ETTTPSKQSN NKYAASSYLS
LTPEQWKSHR SYSCQVTHEG STVEKTVAPT ECS
SEQ ID NO: 107. elotuzumab heavy chain
EVQLVESGGG LVQPGGSLRL SCAASGEDES RYWMSWVRQA PGKGLEWIGE INPDSSTINY
APSLKDKFII SRDNAKNSLY LQMNSLRAED TAVYYCARPD GNYWYFDVWG QGTLVTVSSA
STKGPSVEPL APSSKSTSGG TAALGCLVKD YFPEPVTVSW NSGALTSGVH TFPAVLQSSG
LYSLSSVVTV PSSSLGTQTY ICNVNHKPSN TKVDKKVEPK SCDKTHTCPP CPAPELLGGP
SVELFPPKPK DTLMISRTPE VTCVVVDVSH EDPEVKENWY VDGVEVHNAK TKPREEQYNS
TYRVVSVLTV LHQDWINGKE YKCKVSNKAL PAPIEKTISK AKGQPREPQV YTLPPSRDEL
TKNQVSLTCL VKGFYPSDIA VEWESNGQPE NNYKTTPPVL DSDGSFFLYS KLTVDKSRWQ
QGNVFSCSVM HEALHNHYTQ KSLSLSPGK
SEQ ID NO: 108. elotuzumab light chain
DIQMTQSPSS LSASVGDRVT ITCKASQDVG IAVAWYQQKP GKVPKLLIYW ASTRHTGVPD
RFSGSGSGTD FTLTISSLQP EDVATYYCQQ YSSYPYTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC
SEQ ID NO: 109. daratumumab heavy chain
EVQLLESGGG LVQPGGSLRL SCAVSGFTEN SFAMSWVRQA PGKGLEWVSA ISGSGGGTYY
ADSVKGRFTI SRDNSKNTLY LQMNSLRAED TAVYFCAKDK ILWFGEPVED YWGQGTLVTV
SSASTKGPSV FPLAPSSKST SGGTAALGCL VKDYFPEPVT VSWNSGALTS GVHTFPAVLQ
SSGLYSLSSV VTVPSSSLGT QTYICNVNHK PSNTKVDKRV EPKSCDKTHT CPPCPAPELL
GGPSVFLFPP KPKDTLMISR TPEVTCVVVD VSHEDPEVKF NWYVDGVEVH NAKTKPREEQ
YNSTYRVVSV LTVLHQDWLN GKEYKCKVSN KALPAPIEKT ISKAKGQPRE PQVYTLPPSR
EEMTKNQVSL TCLVKGFYPS DIAVEWESNG QPENNYKTTP PVLDSDGSFF LYSKLTVDKS
RWQQGNVFSC SVMHEALHNH YTQKSLSLSP GK
SEQ ID NO: 110. daratumumab light chain
EIVLTQSPAT LSLSPGERAT LSCRASQSVS SYLAWYQQKP GQAPRLLIYD ASNRATGIPA
RFSGSGSGTD FTLTISSLEP EDFAVYYCQQ RSNWPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC
SEQ ID NO: 111. isatuximab heavy chain
QVQLVQSGAE VAKPGTSVKL SCKASGYTFT DYWMQWVKQR PGQGLEWIGT IYPGDGDTGY
AQKFQGKATL TADKSSKTVY MHLSSLASED SAVYYCARGD YYGSNSLDYW GQGTSVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVELFPPKP KDTLMISRTP EVTCVVVDVS HEDPEVKENW YVDGVEVHNA KTKPREEQYN
STYRVVSVLT VLHQDWLNGK EYKCKVSNKA LPAPIEKTIS KAKGQPREPQ VYTLPPSRDE
LTKNQVSLTC LVKGFYPSDI AVEWESNGQP ENNYKTTPPV LDSDGSFFLY SKLTVDKSRW
QQGNVFSCSV MHEALHNHYT QKSLSLSPGK
SEQ ID NO: 112. isatuximab light chain
DIVMTQSHLS MSTSLGDPVS ITCKASQDVS TVVAWYQQKP GQSPRRLIYS ASYRYIGVPD
RFTGSGAGTD FTFTISSVQA EDLAVYYCQQ HYSPPYTFGG GTKLEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC
SEQ ID NO: 113. 5T4 IGG1-HC-YTE-cleavable-scFv1-sifalimumab
MGVKVLFALI CIAVAEAQVQ LQESGPGLVK PSETLSITCT VSGVSISSYG LHWIRQPPGK
SLEWLGVIWA SGRTNYNPAL KSRVTISGDT SKNQVSLKLS GVTAADTAVY YCAGSNWGYA
MDYWGQGTLV TVSSASTKGP SVEPLAPSSK STSGGTAALG CLVKDYFPEP VTVSWNSGAL
TSGVHTFPAV LQSSGLYSLS SVVTVPSSSL GTQTYICNVN HKPSNTKVDK KAEPKSCDKT
HTCPPCPAPE AAGGPSVELF PPKPKDTLYI TREPEVTCVV VDVSHEDPEV KENWYVDGVE
VHNAKTKPRE EQYNSTYRVV SVLTVLHQDW LNGKEYKCKV SNKALPAPIE KTISKAKGQP
REPQVYTLPP SREEMTKNQV SLTCLVKGFY PSDIAVEWES NGQPENNYKT TPPVLDSDGS
FFLYSKLTVD KSRWQQGNVF SCSVMHEALH NHYTQKSLSL SPGGGGSGGG GSGPLGVRGG
GGSGGGGSEI VLTQSPGTLS LSPGERATLS CRASQSVSST YLAWYQQKPG QAPRLLIYGA
SSRATGIPDR FSGSGSGTDE TLTISRLEPE DFAVYYCQQY GSSPRTFGQG TKVEIKGGGG
SGGGGSGGGG SQVQLVQSGA EVKKPGASVK VSCKASGYTF TSYSISWVRQ APGQGLEWMG
WISVYNGNTN YAQKFQGRVT MTTDTSTSTA YLELRSLRSD DTAVYYCARD PIAAGYWGQG
TLVTVSS**
SEQ ID NO: 114. 5T4 IGG1-HC-YTE-noncleavable-scFv1-sifalimumab
MGVKVLFALI CIAVAEAQVQ LQESGPGLVK PSETLSITCT VSGVSISSYG LHWIRQPPGK
SLEWLGVIWA SGRTNYNPAL KSRVTISGDT SKNQVSLKLS GVTAADTAVY YCAGSNWGYA
MDYWGQGTLV TVSSASTKGP SVEPLAPSSK STSGGTAALG CLVKDYFPEP VTVSWNSGAL
TSGVHTFPAV LQSSGLYSLS SVVTVPSSSL GTQTYICNVN HKPSNTKVDK KAEPKSCDKT
HTCPPCPAPE AAGGPSVELF PPKPKDTLYI TREPEVTCVV VDVSHEDPEV KENWYVDGVE
VHNAKTKPRE EQYNSTYRVV SVLTVLHQDW LNGKEYKCKV SNKALPAPIE KTISKAKGQP
REPQVYTLPP SREEMTKNQV SLTCLVKGFY PSDIAVEWES NGQPENNYKT TPPVLDSDGS
FFLYSKLTVD KSRWQQGNVF SCSVMHEALH NHYTQKSLSL SPGGGGSGGG GSAAGGGGSG
GGGSEIVLTQ SPGTLSLSPG ERATLSCRAS QSVSSTYLAW YQQKPGQAPR LLIYGASSRA
TGIPDRESGS GSGTDFTLTI SRLEPEDFAV YYCQQYGSSP RTFGQGTKVE IKGGGGSGGG
GSGGGGSQVQ LVQSGAEVKK PGASVKVSCK ASGYTFTSYS ISWVRQAPGQ GLEWMGWISV
YNGNTNYAQK FQGRVTMTTD TSTSTAYLEL RSLRSDDTAV YYCARDPIAA GYWGQGTLVT
VSS**
SEQ ID NO: 115. 5T4 IGG1-HC-YTE-IFNalpha-2b 1xG4S
MGVKVLFALI CIAVAEAQVQ LQESGPGLVK PSETLSITCT VSGVSISSYG LHWIRQPPGK
SLEWLGVIWA SGRTNYNPAL KSRVTISGDT SKNQVSLKLS GVTAADTAVY YCAGSNWGYA
MDYWGQGTLV TVSSASTKGP SVEPLAPSSK STSGGTAALG CLVKDYFPEP VTVSWNSGAL
TSGVHTFPAV LQSSGLYSLS SVVTVPSSSL GTQTYICNVN HKPSNTKVDK KAEPKSCDKT
HTCPPCPAPE AAGGPSVELF PPKPKDTLYI TREPEVTCVV VDVSHEDPEV KENWYVDGVE
VHNAKTKPRE EQYNSTYRVV SVLTVLHQDW LNGKEYKCKV SNKALPAPIE KTISKAKGQP
REPQVYTLPP SREEMTKNQV SLTCLVKGFY PSDIAVEWES NGQPENNYKT TPPVLDSDGS
FFLYSKLTVD KSRWQQGNVF SCSVMHEALH NHYTQKSLSL SPGGGGSCDL PQTHSLGSRR
TLMLLAQMRR ISLESCLKDR HDFGFPQEEF GNQFQKAETI PVLHEMIQQI ENLESTKDSS
AAWDETLLDK FYTELYQQLN DLEACVIQGV GVTETPLMKE DSILAVRKYF QRITLYLKEK
KYSPCAWEVV RAEIMRSFSL STNLQESLRS KE**
SEQ ID NO: 116. 5T4 IGG1-HC-YTE-IFNalpha-2b 3xG4S
MGVKVLFALI CIAVAEAQVQ LQESGPGLVK PSETLSITCT VSGVSISSYG LHWIRQPPGK
SLEWLGVIWA SGRTNYNPAL KSRVTISGDT SKNQVSLKLS GVTAADTAVY YCAGSNWGYA
MDYWGQGTLV TVSSASTKGP SVEPLAPSSK STSGGTAALG CLVKDYFPEP VTVSWNSGAL
TSGVHTFPAV LQSSGLYSLS SVVTVPSSSL GTQTYICNVN HKPSNTKVDK KAEPKSCDKT
HTCPPCPAPE AAGGPSVELF PPKPKDTLYI TREPEVTCVV VDVSHEDPEV KENWYVDGVE
VHNAKTKPRE EQYNSTYRVV SVLTVLHQDW LNGKEYKCKV SNKALPAPIE KTISKAKGQP
REPQVYTLPP SREEMTKNQV SLTCLVKGFY PSDIAVEWES NGQPENNYKT TPPVLDSDGS
FFLYSKLTVD KSRWQQGNVF SCSVMHEALH NHYTQKSLSL SPGGGGSGGG GSGGGGSCDL
PQTHSLGSRR TLMLLAQMRR ISLESCLKDR HDFGFPQEEF GNQFQKAETI PVLHEMIQQI
FNLFSTKDSS AAWDETLLDK FYTELYQQLN DLEACVIQGV GVTETPLMKE DSILAVRKYF
QRITLYLKEK KYSPCAWEVV RAEIMRSESL STNLQESLRS KE**
SEQ ID NO: 117. 5T4 IGG1-HC-YTE-RF IFNalpha-2b 1xG4S
MGVKVLFALI CIAVAEAQVQ LQESGPGLVK PSETLSITCT VSGVSISSYG LHWIRQPPGK
SLEWLGVIWA SGRTNYNPAL KSRVTISGDT SKNQVSLKLS GVTAADTAVY YCAGSNWGYA
MDYWGQGTLV TVSSASTKGP SVEPLAPSSK STSGGTAALG CLVKDYFPEP VTVSWNSGAL
TSGVHTFPAV LQSSGLYSLS SVVTVPSSSL GTQTYICNVN HKPSNTKVDK KAEPKSCDKT
HTCPPCPAPE AAGGPSVELF PPKPKDTLYI TREPEVTCVV VDVSHEDPEV KENWYVDGVE
VHNAKTKPRE EQYNSTYRVV SVLTVLHQDW LNGKEYKCKV SNKALPAPIE KTISKAKGQP
REPQVYTLPP SREEMTKNQV SLTCLVKGFY PSDIAVEWES NGQPENNYKT TPPVLDSDGS
FFLYSKLTVD KSRWQQGNVF SCSVMHEALH NRFTQKSLSL SPGGGGSCDL PQTHSLGSRR
TLMLLAQMRR ISLESCLKDR HDFGFPQEEF GNQFQKAETI PVLHEMIQQI FNLESTKDSS
AAWDETLLDK FYTELYQQLN DLEACVIQGV GVTETPLMKE DSILAVRKYF QRITLYLKEK
KYSPCAWEVV RAEIMRSFSL STNLQESLRS KE**
SEQ ID NO: 118. 5T4 IGG1-HC-YTE-RF IFNalpha-2b 3xG4S
MGVKVLFALI CIAVAEAQVQ LQESGPGLVK PSETLSITCT VSGVSISSYG LHWIRQPPGK
SLEWLGVIWA SGRTNYNPAL KSRVTISGDT SKNQVSLKLS GVTAADTAVY YCAGSNWGYA
MDYWGQGTLV TVSSASTKGP SVEPLAPSSK STSGGTAALG CLVKDYFPEP VTVSWNSGAL
TSGVHTEPAV LQSSGLYSLS SVVTVPSSSL GTQTYICNVN HKPSNTKVDK KAEPKSCDKT
HTCPPCPAPE AAGGPSVELF PPKPKDTLYI TREPEVTCVV VDVSHEDPEV KENWYVDGVE
VHNAKTKPRE EQYNSTYRVV SVLTVLHQDW LNGKEYKCKV SNKALPAPIE KTISKAKGQP
REPQVYTLPP SREEMTKNQV SLTCLVKGFY PSDIAVEWES NGQPENNYKT TPPVLDSDGS
FFLYSKLTVD KSRWQQGNVF SCSVMHEALH NRFTQKSLSL SPGGGGSGGG GSGGGGSCDL
PQTHSLGSRR TLMLLAQMRR ISLESCLKDR HDFGFPQEEF GNQFQKAETI PVLHEMIQQI
FNLFSTKDSS AAWDETLLDK FYTELYQQLN DLEACVIQGV GVTETPLMKE DSILAVRKYF
QRITLYLKEK KYSPCAWEVV RAEIMRSESL STNLQESLRS KE**
SEQ ID NO: 119. Anti-5T4 Heavy Chain-YTE-knob-RF-IFNalpha-2a
MGVKVLFALI CIAVAEAQVQ LQESGPGLVK PSETLSITCT VSGVSISSYG LHWIRQPPGK
SLEWLGVIWA SGRTNYNPAL KSRVTISGDT SKNQVSLKLS GVTAADTAVY YCAGSNWGYA
MDYWGQGTLV TVSSASTKGP SVEPLAPCSR STSESTAALG CLVKDYFPEP VTVSWNSGAL
TSGVHTFPAV LQSSGLYSLS SVVTVPSSSL GTKTYTCNVD HKPSNTKVDK RVESKYGPPC
PPCPAPEFLG GPSVFLFPPK PKDTLYITRE PEVTCVVVDV SQEDPEVQEN WYVDGVEVHN
AKTKPREEQF NSTYRVVSVL TVLHQDWING KEYKCKVSNK GLPSSIEKTI SKAKGQPREP
QVYTLPPCQE EMTKNQVSLW CLVKGFYPSD IAVEWESNGQ PENNYKTTPP VLDSDGSFEL
YSRLTVDKSR WQEGNVESCS VMHEALHNRF TQKSLSLSLG AGGGGSGGGG SGGGGSCDLP
QTHSLGSRRT LMLLAQMRKI SLFSCLKDRH DEGFPQEEFG NQFQKAETIP VLHEMIQQIF
NLFSTKDSSA AWDETLLDKF YTELYQQLND LEACVIQGVG VTETPLMKED SILAVRKYFQ
RITLYLKEKK YSPCAWEVVR AEIMRSFSLS TNLQESLRSK E**
SEQ ID NO: 120. LC-5T4
MDMRVPAQLL GLLLLWLPGA KCAIRMTQSP SSESASAGDR VTITCRASEN IYSYLAWYQQ
KPGKSPKLLI YNAKTLAEGV PSRESGSGSG TDFTLTISSL QPEDFATYYC QHHYAIPYTE
GQGTKLEIKR TVAAPSVFIF PPSDEQLKSG TASVVCLLNN FYPREAKVQW KVDNALQSGN
SQESVTEQDS KDSTYSLSST LTLSKADYEK HKVYACEVTH QGLSSPVTKS ENRGEC**
SEQ ID NO: 121. HC-anti-5T4 antibody
MGVKVLFALI CIAVAEAQVQ LQESGPGLVK PSETLSITCT VSGVSISSYG LHWIRQPPGK
SLEWLGVIWA SGRTNYNPAL KSRVTISGDT SKNQVSLKLS GVTAADTAVY YCAGSNWGYA
MDYWGQGTLV TVSSASTKGP SVEPLAPCSR STSESTAALG CLVKDYFPEP VTVSWNSGAL
TSGVHTFPAV LQSSGLYSLS SVVTVPSSSL GTKTYTCNVD HKPSNTKVDK RVESKYGPPC
PPCPAPEFLG GPSVELFPPK PKDTLMISRT PEVTCVVVDV SQEDPEVQEN WYVDGVEVHN
AKTKPREEQF NSTYRVVSVL TVLHQDWLNG KEYKCKVSNK GLPSSIEKTI SKAKGQPREP
QVYTLPPSQE EMTKNQVSLT CLVKGFYPSD IAVEWESNGQ PENNYKTTPP VLDSDGSFEL
YSRLTVDKSR WQEGNVESCS VMHEALHNHY TQKSLSLSLG K**
SEQ ID NO: 122
GGGGS
SEQ ID NO: 123
GGGGSGGGGS
SEQ ID NO: 124
GGGGSGGGGS GGGGS
SEQ ID NO: 125
GGGGSGGGGS AAGGGGSGGG GS
SEQ ID NO: 126. HC-anti-CD38 antibody daratumumab HC-IFNa-2b-Mask
EVQLLESGGG LVQPGGSLRL SCAVSGFTEN SFAMSWVRQA PGKGLEWVSA ISGSGGGTYY
ADSVKGRFTI SRDNSKNTLY LQMNSLRAED TAVYFCAKDK ILWFGEPVED YWGQGTLVTV
SSASTKGPSV FPLAPSSKST SGGTAALGCL VKDYFPEPVT VSWNSGALTS GVHTFPAVLQ
SSGLYSLSSV VTVPSSSLGT QTYICNVNHK PSNTKVDKRV EPKSCDKTHT CPPCPAPELL
GGPSVFLFPP KPKDTLMISR TPEVTCVVVD VSHEDPEVKF NWYVDGVEVH NAKTKPREEQ
YNSTYRVVSV LTVLHQDWLN GKEYKCKVSN KALPAPIEKT ISKAKGQPRE PQVYTLPPSR
EEMTKNQVSL TCLVKGFYPS DIAVEWESNG QPENNYKTTP PVLDSDGSFF LYSKLTVDKS
RWQQGNVFSC SVMHEALHNH YTQKSLSLSP GGGGSGGGGS GGGGSCDLPQ THSLGSRRTL
MLLAQMRRIS LESCLKDRHD FGFPQEEFGN QFQKAETIPV LHEMIQQIEN LESTKDSSAA
WDETLLDKFY TELYQQLNDL EACVIQGVGV TETPLMKEDS ILAVRKYFQR ITLYLKEKKY
SPCAWEVVRA EIMRSFSLST NLQESLRSKE GGGGSGGGGS GPLGLAGGGG SGGGGSEIVL
TQSPGTLSLS PGERATLSCR ASQSVSSTYL AWYQQKPGQA PRLLIYGASS RATGIPDRES
GSGSGTDFTL TISRLEPEDF AVYYCQQYGS SPRTFGQGTK VEIKGGGGSG GGGSGGGGSQ
VQLVQSGAEV KKPGASVKVS CKASGYTFTS YSISWVRQAP GQGLEWMGWI SVYNGNTNYA
QKFQGRVTMT TDTSTSTAYL ELRSLRSDDT AVYYCARDPI AAGYWGQGTL VTVSS
SEQ ID NO: 127. Anti-CD38 antibody isatuximab Heavy Chain-IFNα-2b-
MVQLVQSGAE VAKPGTSVKL SCKASGYTFT DYWMQWVKQR PGQGLEWIGT IYPGDGDTGY
AQKFQGKATL TADKSSKTVY MHLSSLASED SAVYYCARGD YYGSNSLDYW GQGTSVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVELFPPKP KDTLMISRTP EVTCVVVDVS HEDPEVKENW YVDGVEVHNA KTKPREEQYN
STYRVVSVLT VLHQDWLNGK EYKCKVSNKA LPAPIEKTIS KAKGQPREPQ VYTLPPSRDE
LTKNQVSLTC LVKGFYPSDI AVEWESNGQP ENNYKTTPPV LDSDGSFFLY SKLTVDKSRW
QQGNVFSCSV MHEALHNHYT QKSLSLSPGG GGSGGGGSGG GGSCDLPQTH SLGSRRTLML
LAQMRRISLF SCLKDRHDFG FPQEEFGNQF QKAETIPVLH EMIQQIFNLF STKDSSAAWD
ETLLDKFYTE LYQQLNDLEA CVIQGVGVTE TPLMKEDSIL AVRKYRQRIT LYLKEKKYSP
CAWEVVRAEI MRSFSLSTNL QESLRSKEGG GGSGGGGSGP LGLAGGGGSG GGGSEIVLTQ
SPGTLSLSPG ERATLSCRAS QSVSSTYLAW YQQKPGQAPR LLIYGASSRA TGIPDRESGS
GSGTDETLTI SRLEPEDFAV YYCQQYGSSP RTFGQGTKVE IKGGGGSGGG GSGGGGSQVQ
LVQSGAEVKK PGASVKVSCK ASGYTFTSYS ISWVRQAPGQ GLEWMGWISV YNGNTNYAQK
FQGRVTMTTD TSTSTAYLEL RSLRSDDTAV YYCARDPIAA GYWGQGTLVT VSS

1. A prodrug comprising a human interferon (IFN) agonist polypeptide, a masking moiety, and a carrier moiety, wherein
the masking moiety comprises an antigen-binding fragment of an antibody that binds to the human interferon agonist polypeptide and inhibits a biological activity of the human interferon agonist polypeptide,
the human interferon agonist polypeptide is interferon alpha (IFNα) and is fused to the carrier moiety, and
the masking moiety is fused to the human interferon agonist polypeptide or to the carrier moiety, optionally through a peptide linker.

2. A prodrug comprising a human interferon (IFN) agonist polypeptide, a masking moiety, and a carrier moiety, wherein
the masking moiety binds to the human interferon agonist polypeptide and inhibits a biological activity of the human interferon agonist polypeptide,
the human interferon agonist polypeptide is fused to the carrier moiety, and
the masking moiety is fused to the human interferon agonist polypeptide or to the carrier moiety, optionally through a peptide linker.

3. The prodrug of claim 1, wherein the IFNα is IFNα-2a, IFNα-2b, or an analog thereof.

4. The prodrug of claim 2, wherein the IFN is IFNα-2a, IFNα-2b, IFNγ, or an analog thereof.

5. The prodrug of claim 4, wherein the IFNγ comprises an amino acid sequence selected from SEQ ID NO: 5, 6, and 7, or at least 90% identical to SEQ ID NO: 5, 6, or 7.

6. The prodrug of any one of claims 1-4, wherein the INFα-2a comprises an amino acid sequence selected from SEQ ID NO: 65 or at least 95% identical to SEQ ID NO: 65.

7. The prodrug of any one of claims 1-4, wherein the INFα-2b comprises an amino acid sequence selected from SEQ ID NO: 66 or at least 95% identical to SEQ ID NO: 66.

8. The prodrug of any of claims 1-7, wherein the IFN agonist polypeptide is fused to the carrier through a non-cleavable peptide linker and the masking moiety is fused to the carrier through a cleavable peptide linker or a non-cleavable peptide linker.

9. The prodrug of any of claims 1-7, wherein the IFN agonist polypeptide is fused to the carrier through a non-cleavable peptide linker or a cleavable linker and the masking moiety is fused to the carrier through a non-cleavable peptide linker.

10. The prodrug of any of claims 1-7, wherein the carrier comprises an antibody Fc domain with a first Fc polypeptide chain with knob mutations and a second Fc polypeptide chain with hole mutations; wherein the IFN agonist polypeptide is fused to the first the carrier through a cleavable peptide linker and the masking moiety is fused to the second Fc polypeptide chain through a non-cleavable peptide linker.

11. The prodrug of any one of claims 1-7, wherein the carrier moiety is an antibody Fc domain or an antibody comprising knobs-into-holes mutations, and wherein
the human IFN agonist polypeptide and its masking moiety are fused to different polypeptide chains of the antibody Fc domain or to the different heavy chains of the antibody.

12. The prodrug of claim 1-7, wherein the carrier is an antibody, the prodrug comprises two IFN agonist polypeptides fused to the C-terminus of the two heavy chains of the antibody through non-cleavable peptide linkers and two masking moieties that are fused to the two IFN agonist polypeptide through cleavable peptide linkers.

13. The prodrug of any one of claim 1-11, wherein the peptide linker is a cleavable peptide linker comprising a substrate sequence of urokinase-type plasminogen activator (uPA), matrix metallopeptidase (MT1-MMP), matrix metallopeptidase 2 (MMP2), MMP9, matriptase, legumain, plasmin, TMPRSS-3/4, cathepsin, caspase, human neutrophil elastase, beta-secretase, or PSA, or (i) both uPA and MMP2, (ii) both uPA and MMP9, or (iii) matriptase, MMP2 and MMP9.

14. The prodrug of any one of claims 8-13, wherein the cleavable peptide linker comprises an amino acid sequence selected from SEQ ID NOs: 26-45.

15. The prodrug of any one of claims 8-13, wherein the non-cleavable peptide linker comprises an amino acid sequence selected from SEQ ID NOs: 122-125.

16. The prodrug of any one of claims 8-13, wherein the cleavable peptide linker is cleavable by one or more proteases located at a tumor site or its surrounding environment, and the cleavage leads to activation of the prodrug at the tumor site or surrounding environment.

17. The prodrug of any one of the preceding claims, wherein the masking moiety inhibits the binding of the IFN agonist polypeptide to an IFN receptor.

18. The prodrug of any one of the preceding claims, wherein the masking moiety comprises a single chain fragment variable (scFv) comprising a heavy chain variable domain with an amino acid sequence as shown SEQ ID NO: 1 and a light chain variable domain with an amino acid sequence as shown in SEQ ID NO: 2, or a heavy chain variable domain with an amino acid sequence as shown SEQ ID NO: 3 and a light chain variable domain with an amino acid sequence as shown in SEQ ID NO: 4.

19. The prodrug of any one of claim 2 or 4-18, wherein the masking moiety is selected from interferon gamma receptor 1 extracellular domain (IFNGR1-ECD) or a functional analog thereof, or an antibody or a binding fragment thereof which binds to IFNγ.

20. The prodrug of claim 19, wherein the masking moiety comprises IFNGR1-ECD or a functional analog thereof, and optionally wherein the IFNGR1-ECD comprises an amino acid sequence selected from SEQ ID NOs: 8 and 9.

21. The prodrug of any of any one of the preceding claims, wherein the carrier moiety is an antibody Fc domain, an antibody, or an antigen-binding fragment of an antibody.

22. The prodrug of any one of the preceding claims, wherein the carrier moiety comprises an antibody or antigen-binding fragment thereof that binds to an antigen expressed on the surface of a tumor cell, a cell in the tumor microenvironment, a cancer cell, or an immune cell.

23. The prodrug of claim 21, wherein the immune cell is selected from an NK cell, a T cell, a B cell, and a macrophage.

24. The prodrug of any one of claims 21-23, wherein the carrier moiety comprises an antibody or antigen-binding fragment thereof that binds to an antigen selected from PD-1, LAG-3, TIGIT, SIRPα, ILT2, CD206, NKD2G, CTLA-4, CD8, NKG2A, CD16a, CD38, BCMA, cell-surface glycoprotein CD2 subset 1 (CS1), PD-L1, CD47, CMET, EGFR, ROR1, TROP-2, HER2, CLDN18.2, and VEGFR2.

25. The prodrug of any one of claims 21-23, wherein the carrier moiety comprises an antibody or antigen-binding fragment thereof that binds to PD-1, and wherein the antibody comprises a light chain variable domain with an amino acid sequence of SEQ ID NO: 10 and a heavy chain variable domain with an amino acid sequence of SEQ ID NO: 11, or a light chain variable domain with an amino acid sequence of SEQ ID NO: 12 and a heavy chain variable domain with an amino acid sequence of SEQ ID NO: 13.

26. The prodrug of any one of claims 21-23, wherein the carrier moiety comprises an antibody or antigen-binding fragment comprising:
i) a heavy chain variable domain with an amino acid sequence of SEQ ID NO: 97 or at least 95% identical to SEQ ID NO: 97, and a light chain with an amino acid sequence of SEQ ID NO: 98 or at least 95% identical to SEQ ID NO: 98; or
(ii) a heavy chain variable domain with an amino acid sequence of SEQ ID NO: 99 or at least 95% identical to SEQ ID NO: 99, and a light chain with an amino acid sequence of SEQ ID NO:100 or at least 95% identical to SEQ ID NO: 100; or
(iii) a heavy chain variable domain with an amino acid sequence of SEQ ID NO: 101 or at least 95% identical to SEQ ID NO: 101, and a light chain with an amino acid sequence of SEQ ID NO: 102 or at least 95% identical to SEQ ID NO: 102.

27. The prodrug of any one of claim 1-4, 6-11, 13-17, or 21-23, wherein the prodrug comprises a first polypeptide chain and a second polypeptide chain; wherein said first polypeptide chain comprises an amino acid sequence selected from SEQ ID NOs: 67, 68, 69, and 70 or at least 95% identical to SEQ ID NO: 67, 68, 69, or 70, and said second polypeptide chain comprises an amino acid sequence selected from SEQ ID NOs: 71 and 72 or at least 95% identical to SEQ ID NO: 71 or 72.

28. The prodrug of any one of claim 1-4, 6-11, 13-17, or 21-23, wherein the prodrug comprises a first polypeptide chain and a second polypeptide chain; wherein said first polypeptide chain comprises an amino acid sequence selected from SEQ ID NOs: 73, 74, 75, and 76 or at least 95% identical to SEQ ID NO: 73, 74, 75, or 76, and said second polypeptide chain comprises an amino acid sequence selected from SEQ ID NO: 77, 78 or at least 95% to SEQ ID NO: 77 or 78.

29. The prodrug of any one of claim 1-4, 6-11, 13-17, or 21-23, wherein the prodrug comprises two identical light chains, a first heavy chain polypeptide chain and a second heavy chain polypeptide chain, wherein said light chain comprises an amino acid sequence of SEQ ID NO:46 or at least 95% identical to SEQ ID NO: 46, said first heavy chain polypeptide chain comprises an amino acid sequence selected from SEQ ID NOs: 79, 80, 81, and 82 or at least 98% identical to SEQ ID NO: 79, 80, 81, or 82, and said second polypeptide chain comprises an amino acid sequence selected from SEQ ID NOs:83 and 84 or at least 98% identical to SEQ ID NO: 83 or 84.

30. The prodrug of any one of claim 1-4, 6-11, 13-17, or 21-23, wherein the prodrug comprises two identical light chains, a first heavy chain polypeptide chain and a second heavy chain polypeptide chain; wherein said light chain comprises an amino acid sequence of SEQ ID NO: 53 or at least 95% identical to SEQ ID NO: 53, said first heavy chain polypeptide chain comprises an amino acid sequence selected from SEQ ID NOs: 85, 86, 87, and 88 or at least 98% identical to SEQ ID NO: 85, 86, 87, or 88, and said second polypeptide chain comprises an amino acid sequence selected from SEQ ID NOs: 89 and 90 or at least 98% identical to SEQ ID NO: 89 or 90.

31. The prodrug of any one of claim 1-4, 6-11, 13-17, or 21-23, wherein the prodrug comprises two identical light chains, a first heavy chain polypeptide chain and a second heavy chain polypeptide chain; wherein said light chain comprises an amino acid sequence of SEQ ID NO:120 or at least 95% identical to 120, said first heavy chain polypeptide chain comprises an amino acid sequence selected from SEQ ID NOs: 113 and 114 or at least 98% identical to SEQ ID NO:113 or 114, and said second polypeptide chain comprises an amino acid sequence selected from SEQ ID NOs:115, 116, 117, 118, and 119 or at least 98% identical to SEQ ID NO: 115, 116, 117, 118, or 119.

32. The prodrug of any one of claim 1-4, 6-11, 13-17, or 21-23, wherein the prodrug comprises two identical light chains, a two identical heavy chain polypeptide chains; wherein said light chain comprises an amino acid sequence of SEQ ID NO: 94 or at least 95% identical to SEQ ID NO: 94, said heavy chain polypeptide chain comprises an amino acid sequence of SEQ ID NO: 126 or at least 98% identical to SEQ ID NO: 126.

33. The prodrug of any one of claim 1-4, 6-11, 13-17, or 21-23, wherein the prodrug comprises two identical light chains, a two identical heavy chain polypeptide chains; wherein said light chain comprises an amino acid sequence of SEQ ID NO: 96 or at least 95% identical to SEQ ID NO: 96, said heavy chain polypeptide chain comprises an amino acid sequence of SEQ ID NO: 127 or at least 98% identical to SEQ ID NO: 127.

34. A pharmaceutical composition comprising the prodrug of any one of claims 1-33 and a pharmaceutically acceptable excipient.

35. A polynucleotide or polynucleotides encoding the prodrug of any one of claims 1-33.

36. An expression vector or vectors comprising the polynucleotide or polynucleotides of claim 35.

37. A host cell comprising the vector(s) of claim 36.

38. The host cell of claim 37, wherein the gene(s) encoding matriptase, uPA, MMP-2, and/or MMP-9 are knocked out in the host cell.

39. A method of making the prodrug of any one of claims 1-33, comprising
culturing the host cell of claim 37 or 38 under conditions that allow expression of the prodrug, wherein the host cell is a mammalian cell, and
isolating the prodrug.

40. A method of treating a cancer or an infectious disease or stimulating the immune system in a patient in need thereof, comprising administering to the patient a therapeutically effective amount of the pharmaceutical composition of claim 34.

41. A prodrug of any one of claims 1-33 for use in treating a cancer or an infectious disease or stimulating the immune system in a patient in need thereof.

42. Use of a prodrug of any one of claims 1-33 for the manufacture of a medicament for treating a cancer or an infectious disease or stimulating the immune system in a patient in need thereof.

43. The method of claim 40, the prodrug for use of claim 41, or the use of claim 42, wherein the patient has a virus infection, or a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer.
